SEQUENCE LISTING (76 sequences)

SEQ ID NO: 1 - DNA, 2117 bp, Artificial Sequence
  SVF-1 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 2 - DNA, 2780 bp, Artificial Sequence
  SVF-2 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 3 - DNA, 2849 bp, Artificial Sequence
  SVF-3 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagggaagcggagctactaacttcagcctgctgaagcaggctggagacgtggaggagaaccctggacctatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 4 - DNA, 1520 bp, Artificial Sequence
  SVF-4 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtgatgatctaga

SEQ ID NO: 5 - DNA, 2048 bp, Artificial Sequence
  SVF-5 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 6 - DNA, 2711 bp, Artificial Sequence
  SVF-6 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 7 - DNA, 1451 bp, Artificial Sequence
  SVF-7 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaaggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtgatgatctaga

SEQ ID NO: 8 - DNA, 3344 bp, Artificial Sequence
  SVF-8 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagtgatgatctaga

SEQ ID NO: 9 - DNA, 3620 bp, Artificial Sequence
  SVF-9 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagggaagcggagctactaacttcagcctgctgaagcaggctggagacgtggaggagaaccctggacctatgagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagggaagcggagctactaacttcagcctgctgaagcaggctggagacgtggaggagaaccctggacctatgagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagggaagcggagctactaacttcagcctgctgaagcaggctggagacgtggaggagaaccctggacctatgagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagggaagcggagctactaacttcagcctgctgaagcaggctggagacgtggaggagaaccctggacctatgagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagtgatgatctaga

SEQ ID NO: 10 - DNA, 788 bp, Artificial Sequence
  SVF-10 wild-type DNA sequence
ggatccgcaccatggccttaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgatgatctaga

SEQ ID NO: 11 - DNA, 689 bp, Artificial Sequence
  SVF-11 wild-type DNA sequence
ggatccgcaccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtgatgatctaga

SEQ ID NO: 12 - DNA, 1286 bp, Artificial Sequence
  SVF-12 wild-type DNA sequence
ggatccgcaccatggcctctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaatgatgatctaga

SEQ ID NO: 13 - DNA, 2118 bp, Artificial Sequence
  SVF-1 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacccatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataataatagtctaga

SEQ ID NO: 14 - DNA, 2781 bp, Artificial Sequence
  SVF-2 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataataatagtctaga

SEQ ID NO: 15 - DNA, 2850 bp, Artificial Sequence
  SVF-3 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcagggcagcggagcaaccaactttagtctgctgaaacaggccggagacgtggaagaaaaccccggacccatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataataatagtctaga

SEQ ID NO: 16 - DNA, 1524 bp, Artificial Sequence
  SVF-4 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcagtaataatagtctaga

SEQ ID NO: 17 - DNA, 2049 bp, Artificial Sequence
  SVF-5 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataataatagtctaga

SEQ ID NO: 18 - DNA, 2712 bp, Artificial Sequence
  SVF-6 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcaggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataataatagtctaga

SEQ ID NO: 19 - DNA, 1455 bp, Artificial Sequence
  SVF-7 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaaggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcagtaataatagtctaga

SEQ ID NO: 20 - DNA, 3348 bp, Artificial Sequence
  SVF-8 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagtccaggtctgagagcaagaagaataggggaggaagggaggagatcctggagcagtgggtgggagcacgcaagaagctggaggagctggagcgggacctgagaaagatcaagaagaagatcaagaagctggaagaggagaacccctggctgggcaatatcaagggcatcctgggcaagaaggatagggagggcgagggagcaccacctgcaaagagggcaagggcagaccagatggaggtggattccggacctaggaagcggcccttccggggagagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggaccaagggtgggaggagtgaatcctctggagggaggaacaaggggagcaccaggaggaggcttcgtgcctagcatgcagggcgtgcctgagtccccatttgccaggaccggagagggcctggacgtgagaggaaaccagggcttcccatgggacatcctgtttccagccgatccacccttcagcccacagtcctgcaggcctcagagccggtccgagtctaagaagaatagaggcggaagggaggaggtgctggagcagtgggtgaacggcagaaagaagctggaagaactggagagggagctgagaagggcccgcaagaagatcaagaagttagaggacgataatccttggctgggcaatgtgaaaggcatcctgggcaagaaggacaaggatggagagggagcacctccagcaaagagggcacgcacagaccagatggagatcgatagcggaccaaggaagcggcccctgagaggaggctttaccgacagggagaggcaggatcaccgccggagaaaggccctgaagaacaagaagaagcagctgtccgccggaggcaagagcctgtccaaagaagaggaggaggagctgaagcggctgacaagagaggacgaggagcggaagaaggaggagcacggaccaagccggctgggagtgaatccaagcgagggaggacctagaggcgcccctggcggaggcttcgtgccttccatgcagggcatcccagagtctaggtttaccaggacaggcgaaggcctggacgtgcggggctccagaggctttccccaggacatcctgttcccttctgatccccctttttctccacagagctgtagaccacagtctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagcggcccagaaccgatcagatggaggtggatagcggccctggcaagaggccacacaagtccggcttcaccgacaaggagagggaggaccataggcgccggaaggccctggagaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggaccaaggggagcacctgggggcggcttcgtgcctcagatggcaggagtgccagagtcccctttttctcggacaggcgagggcctggatatcagaggaacccagggattcccatgggtgtccccatctcctccacagcagaggctgccactgctggagtgcaccccacagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagtaataatagtctaga

SEQ ID NO: 21 - DNA, 3624 bp, Artificial Sequence
  SVF-9 codon optimized DNA sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagggcagcggagccacaaacttttccctgctgaagcaggcaggcgacgtggaggagaatccaggacctatgtccaggtctgagagcaagaagaataggggaggaagggaggagatcctggagcagtgggtgggagcacgcaagaagctggaggagctggagcgggacctgagaaagatcaagaagaagatcaagaagctggaagaggagaacccctggctgggcaatatcaagggcatcctgggcaagaaggatagggagggcgagggagcaccacctgcaaagagggcaagggcagaccagatggaggtggattccggacctaggaagcggcccttccggggagagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggaccaagggtgggaggagtgaatcctctggagggaggaacaaggggagcaccaggaggaggcttcgtgcctagcatgcagggcgtgcctgagtccccatttgccaggaccggagagggcctggacgtgagaggaaaccagggcttcccatgggacatcctgtttccagccgatccacccttcagcccacagtcctgcaggcctcagggctccggcgccaccaatttctctctgctgaagcaggccggcgatgtggaagaaaaccctggaccaatgagccggtccgagtctaagaagaatagaggcggaagggaggaggtgctggagcagtgggtgaacggcagaaagaagctggaagaactggagagggagctgagaagggcccgcaagaagatcaagaagttagaggacgataatccttggctgggcaatgtgaaaggcatcctgggcaagaaggacaaggatggagagggagcacctccagcaaagagggcacgcacagaccagatggagatcgatagcggaccaaggaagcggcccctgagaggaggctttaccgacagggagaggcaggatcaccgccggagaaaggccctgaagaacaagaagaagcagctgtccgccggaggcaagagcctgtccaaagaagaggaggaggagctgaagcggctgacaagagaggacgaggagcggaagaaggaggagcacggaccaagccggctgggagtgaatccaagcgagggaggacctagaggcgcccctggcggaggcttcgtgccttccatgcagggcatcccagagtctaggtttaccaggacaggcgaaggcctggacgtgcggggctccagaggctttccccaggacatcctgttcccttctgatccccctttttctccacagagctgtagaccacagggcagcggagcaaccaacttctccctgctgaagcaagccggcgatgtggaggagaatccaggacctatgtctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagcggcccagaaccgatcagatggaggtggatagcggccctggcaagaggccacacaagtccggcttcaccgacaaggagagggaggaccataggcgccggaaggccctggagaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggaccaaggggagcacctgggggcggcttcgtgcctcagatggcaggagtgccagagtcccctttttctcggacaggcgagggcctggatatcagaggaacccagggattcccatgggtgtccccatctcctccacagcagaggctgccactgctggagtgcaccccacagggctctggagccacaaactttagcctgctgaagcaggccggcgacgtggaagaaaacccaggacccatgagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagtaataatagtctaga

SEQ ID NO: 22 - DNA, 792 bp, Artificial Sequence
  SVF-10 codon optimized sequence
ggatccgccaccatggctctgaagtataacgagaatgggactattaccgatgctgtggactgtgctctggaccctctgagtgagactaaatgtaccctgaagtcctttacagtggagaagggcatctatcagacctctaacttccgggtgcagcctacagagagcatcgtgcgcttccctaacatcaccaatctgtgcccattcggcgaggtgtttaacgccacacggttcgccagcgtgtacgcctggaatcggaagagaatctccaactgcgtggccgactattctgtgctgtacaattccgcctctttcagcacctttaagtgctatggcgtgtccccaaccaagctgaatgacctgtgcttcacaaacgtgtacgccgactcttttgtgatccggggcgatgaggtgagacagatcgcaccaggacagaccggcaagatcgcagactacaattataagctgcctgacgacttcaccggctgcgtgatcgcctggaactctaacaatctggatagcaaagtgggcggcaactacaattatctgtaccggctgttcagaaagtccaacctgaagccatttgagagggacatcagcaccgagatctaccaggccggctccacaccctgcaatggcgtggagggcttcaactgttattttcccctgcagagctacggctttcagcctaccaatggcgtgggctatcagccataccgcgtggtggtgctgtccttcgagctgctgcacgcaccagcaaccgtgtgcggacccaagaagtaataatagtctaga

SEQ ID NO: 23 - DNA, 693 bp, Artificial Sequence
  SVF-11 codon optimized DNA sequence
ggatccgccaccatggccgatagcaacggcaccatcacagtggaggagctgaagaagctgctggagcagtggaacctggtcatcggcttcctgtttctgacatggatctgtctgctgcagttcgcctatgccaacaggaatcgctttctgtacatcatcaagctgatcttcctgtggctgctgtggccagtgaccctggcatgcttcgtgctggccgccgtgtatcggatcaactggatcacaggcggcatcgccatcgccatggcctgtctggtgggcctgatgtggctgtcctactttatcgcctctttcagactgtttgcccggaccagatccatgtggtctttcaaccccgagacaaatatcctgctgaacgtgcctctgcacggcaccatcctgacaaggccactgctggagtccgagctggtcatcggagccgtgatcctgaggggacacctgaggatcgcaggacaccacctgggccgctgtgacatcaaggatctgcctaaggagatcaccgtggccacaagccggaccctgtcctactataagctgggagcatctcagagagtggcaggcgattctggcttcgccgcctatagcaggtaccgcatcggcaattacaagctgaacaccgaccacagctcctctagcgataacatcgccctgctggtgcagtaataatagtctaga

SEQ ID NO: 24 - DNA, 1286 bp, Artificial Sequence
  SVF-12 codon optimized sequence
ggatccgcaccatggcaagcgacaatggaccacagaatcagaggaacgcaccaagaatcactttcggcggcccaagcgactcaaccggcagcaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaataaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggcagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgagccggatcggaatggaggtgaccccaagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctcaaaacagctgcagcagtcaatgtcaagcgccgattcaactcaggcataatgatgatctaga

SEQ ID NO: 25 - protein, 697 aa, Artificial Sequence
  SVF-1 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 26 - protein, 918 aa, Artificial Sequence
  SVF-2 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 941 aa, Artificial Sequence
  SVF-3 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 28 - protein, 499 aa, Artificial Sequence
  SVF-4 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 29 - protein, 674 aa, Artificial Sequence
  SVF-5 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 30 - protein, 895 aa, Artificial Sequence
  SVF-6 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 31 - protein, 476 aa, Artificial Sequence
  SVF-7 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 32 - protein, 1107 aa, Artificial Sequence
  SVF-8 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 33 - protein, 1199 aa, Artificial Sequence
  SVF-9 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSGATNFSLLKQAGDVEENPGPMSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 34 - protein, 255 aa, Artificial Sequence
  SVF-10 polypeptide sequence
MALKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 35 - protein, 222 aa, Artificial Sequence
  SVF-11 polypeptide sequence
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 36 - protein, 420 aa, Artificial Sequence
  SVF-12 polypeptide sequence
MASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 37 - DNA, 66 bp, Artificial Sequence
  P2A autocatalytic peptide cleavage site DNA sequence
ggaagcggagctactaacttcagcctgctgaagcaggctggagacgtggaggagaaccctggacct

SEQ ID NO: 38 - protein, 22 aa, Artificial Sequence
  P2A autocatalytic peptide cleavage site polypeptide sequence
GSGATNFSLLKQAGDVEENPGP

SEQ ID NO: 39 - DNA, 3335 bp, Artificial Sequence
  OC2.2 codon optimized DNA sequence
ggatccgcaccatggcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 40 - DNA, 3386 bp, Artificial Sequence
  OC2.3 codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 41 - protein, 1103 aa, Artificial Sequence
  OC2.2 polypeptide sequence
MARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 42 - protein, 1120 aa, Artificial Sequence
  OC2.3 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 43 - DNA, 54 bp, Artificial Sequence
  IgE leader sequence
atggactggacctggattctgttcctggtggccgccgccacacgggtgcacagc

SEQ ID NO: 44 - protein, 18 aa, Artificial Sequence
  IgE leader polypeptide sequence
MDWTWILFLVAAATRVHS

SEQ ID NO: 45 - DNA, 1314 bp, Artificial Sequence
  RBDv2 (tandem repeat single chain dimer) codon optimized DNA sequence
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaag

SEQ ID NO: 46 - protein, 438 aa, Artificial Sequence
  RBDv2 (tandem repeat single chain dimer) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 47 - DNA, 1314 bp, Artificial Sequence
  RBDv2 (E501Y) codon optimized DNA sequence
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 48 - DNA, 1314 bp, Artificial Sequence
  RBDv2 (N439K, N501Y) codon optimized DNA sequence
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 49 - DNA, 1314 bp, Artificial Sequence
  RBDv2 (K417N, E484K, N501Y) codon optimized DNA sequence
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaacctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaacctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 50 - DNA, 1314 bp, Artificial Sequence
  RBDv2 (K417N, N439K, E484K, N501Y codon optimized DNA sequence
cgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaag

SEQ ID NO: 51 - DNA, 3816 bp, Artificial Sequence
  SARS-CoV-2 S protein (K986P, V987P) codon optimized DNA sequence
ttcgtctttctggtgctgctgcctctggtcagctcccagtgcgtgaacctgaccacaaggacacagctgccccctgcctatacaaattccttcactcggggcgtgtactatcctgacaaagtgtttagatctagtgtgctgcactccacacaggatctgtttctgccattcttttctaacgtgacttggttccacgccatccacgtgagcgggaccaatggaacaaagaggttcgacaatccagtgctgccctttaacgatggcgtgtacttcgccagtaccgagaagtcaaacatcattcgcgggtggatctttggaactaccctggacagcaaaacccagtccctgctgatcgtgaacaatgccacaaacgtggtcatcaaggtgtgcgaattccagttttgtaatgatcccttcctgggcgtgtactatcataagaacaacaagtcttggatggagagtgaatttcgcgtgtattcaagcgccaacaattgcacatttgagtacgtgagccagcctttcctgatggacctggaaggcaagcaggggaatttcaaaaacctgcgggagttcgtgtttaagaatattgatggctacttcaagatctactctaaacacacccccatcaacctggtgcgggacctgcctcaggggttcagtgccctggagcctctggtggatctgccaatcggaattaacatcacccggtttcagacactgctggccctgcatagatcctacctgacaccaggcgactcctctagtggctggactgctggggccgctgcctactatgtggggtatctgcagccccggaccttcctgctgaaatacaacgagaatggaactattaccgacgctgtggattgcgccctggaccccctgtccgaaactaagtgtaccctgaaatcttttaccgtggagaagggaatctatcagacaagcaatttcagggtgcagccaactgaatccattgtgcgctttccaaatatcaccaacctgtgcccctttggcgaggtgttcaacgctacaagattcgccagcgtgtacgcttggaataggaagcgcatcagcaactgcgtggccgactattccgtgctgtacaactccgcttctttcagtacctttaagtgctatggagtgtcacccaccaaactgaatgacctgtgctttacaaacgtgtacgccgattccttcgtgattaggggcgacgaggtgcgccagatcgctcctggacagaccggcaagattgctgactacaattataaactgccagacgatttcacaggctgcgtgatcgcctggaactctaacaatctggatagtaaagtgggcgggaactacaattatctgtacaggctgtttcgcaagagcaatctgaaacccttcgagcgggacattagtactgaaatctaccaggccggctcaaccccttgcaatggggtggagggcttcaactgttatttcccactgcagtcctacgggttccagcccacaaacggagtgggctatcagccttacagagtggtggtgctgtcttttgaactgctgcacgctccagctacagtgtgcggacccaagaaaagtactaatctggtgaagaacaaatgcgtgaacttcaacttcaacggactgacagggactggagtgctgaccgagagcaacaagaaattcctgccatttcagcagttcggaagggacatcgccgatacaactgacgctgtgcgcgacccccagaccctggaaattctggatatcacaccttgctcattcggaggcgtgagcgtgattacccccggcaccaatacatccaaccaggtggccgtgctgtatcaggacgtgaattgtacagaggtgccagtggctatccacgccgatcagctgactcccacctggcgggtgtacagcactggctccaacgtgtttcagaccagagccggatgcctgattggcgctgagcatgtgaacaatagttatgaatgcgacattccaatcggcgccgggatctgtgcttcataccagacacagactaactctccccggagagccaggagtgtggcttcacagagcatcattgcctataccatgtcactgggggccgaaaatagcgtggcttactctaacaatagtattgctatccccacaaacttcactatttctgtgaccacagagatcctgcccgtgagcatgaccaagacatctgtggactgcaccatgtatatttgtggcgattcaacagaatgcagcaacctgctgctgcagtacggaagcttttgtacacagctgaatcgcgccctgactggcatcgctgtggagcaggacaaaaacactcaggaagtgttcgcccaggtgaagcagatctacaaaaccccacccatcaaggactttgggggcttcaacttcagccagattctgcctgatccatcaaagcccagcaaaaggtcctttatcgaggacctgctgttcaacaaggtgaccctggctgatgccggcttcatcaaacagtatggggattgcctgggagacattgctgcccgcgacctgatctgtgcccagaagtttaatgggctgaccgtgctgcctccactgctgacagatgaaatgattgcccagtacacatctgctctgctggccgggactatcaccagtggatggactttcggagccggcgctgccctgcagattccttttgccatgcagatggcttataggttcaacgggatcggagtgacccagaatgtgctgtacgagaaccagaagctgatcgccaatcagtttaacagcgccattggcaaaatccaggactccctgtcaagcactgcttctgccctggggaaactgcaggatgtggtgaatcagaacgctcaggccctgaataccctggtgaagcagctgtcctctaacttcggggccattagttcagtgctgaatgatatcctgtccaggctggacccccctgaggctgaagtgcagattgaccggctgatcacaggaagactgcagagcctgcagacctacgtgacacagcagctgattagggctgccgaaatccgcgcttccgccaatctggctgccaccaagatgtctgagtgcgtgctggggcagagtaagcgcgtggacttttgtggcaaagggtatcacctgatgtccttcccccagtctgcccctcacggagtggtgtttctgcatgtgacctacgtgcctgctcaggagaagaacttcactaccgctccagccatctgccacgatggcaaagcccattttccccgggaaggcgtgttcgtgtccaacgggacccattggtttgtgacacagagaaatttctacgagcctcagatcattacaactgacaataccttcgtgagcggaaactgtgacgtggtcatcggcatcgtgaacaataccgtgtacgatcctctgcagccagagctggacagctttaaggaggaactggataagtacttcaaaaatcacacctcccccgacgtggatctgggcgacattagtgggatcaatgcctcagtggtgaacattcagaaggagatcgacagactgaacgaagtggctaaaaatctgaacgagagcctgattgatctgcaggagctgggcaagtatgaacagtacatcaaatggccttggtatatttggctgggattcatcgccggcctgattgctatcgtgatggtgactatcatgctgtgctgtatgacctcttgctgtagttgcctgaagggctgctgttcatgtgggagctgctgtaaattcgacgaggacgattccgaaccagtgctgaagggcgtgaaactgcactacact

SEQ ID NO: 52 - protein, 438 aa, Artificial Sequence
  RBDv2 (E501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 53 - protein, 438 aa, Artificial Sequence
  RBDv2 (N439K, N501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 54 - protein, 438 aa, Artificial Sequence
  RBDv2 (K417N, E484K, N501Y) polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 55 - protein, 438 aa, Artificial Sequence
  RBDv2 (K417N, N439K, E484K, N501Y polypeptide sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 56 - protein, 1272 aa, Artificial Sequence
  SARS-CoV-2 S protein (K986P, V987P) polypeptide sequence
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 57 - DNA, 3386 bp, Artificial Sequence
  OC2.3 (N501Y) codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 58 - DNA, 3386 bp, Artificial Sequence
  OC2.3 (N439K, N501Y) codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 59 - DNA, 3386 bp, Artificial Sequence
  OC2.3 (K417N, E484K, N501Y) codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaacctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaacctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 60 - DNA, 3386 bp, Artificial Sequence
  OC2.3 (K417N, N439K, E484K, N501Y) codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaacatcgctgactacaattataaactgcccgacgatttcaccggctgcgtgatcgcctggaacagcaacaagctggattccaaagtgggcgggaactacaattatctgtaccggctgttcagaaagtccaatctgaaaccctttgagagggacatcagtactgaaatctaccaggccgggtcaaccccttgcaatggggtgaagggattcaactgttactttccactgcagagttatggatttcagcccacctatggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaacattgctgactataattacaaactgccagacgatttcactggatgcgtgattgcctggaactctaacaagctggatagtaaagtgggaggcaactataattacctgtataggctgttccgcaagtctaatctgaaaccatttgagcgggacatcagcaccgagatctaccaggccggctccaccccctgcaatggagtgaagggcttcaactgctattttccactgcagagctatgggtttcagcccacatatggggtgggataccagccttatagagtggtggtgctgtccttcgaactgctgcatgcccctgctacagtgtgcggcccaaagaaatctaccaacctggtcaagaacaagggaagcggagctactaacttcagcctgctgaagcaggctggagacgtggaggagaaccctggacctatggccgattccaacggaactattaccgtggaggaactgaagaaactgctggagcagtggaacctggtcatcggcttcctgtttctgacctggatttgtctgctgcagttcgcctacgctaaccggaacagatttctgtacatcattaagctgatcttcctgtggctgctgtggcccgtgactctggcctgcttcgtgctggccgccgtgtacaggatcaactggattaccgggggaatcgccattgctatggcctgtctggtgggcctgatgtggctgtcatacttcatcgccagcttccgcctgtttgctaggacacgcagcatgtggtccttcaaccctgagactaatatcctgctgaacgtgccactgcacggcaccattctgacacggcccctgctggagtccgaactggtcatcggggccgtgattctgaggggacatctgcgcatcgctgggcaccatctgggaagatgcgacatcaaggatctgcctaaagaaattactgtggccacctcaaggacactgagctactataagctgggagctagccagagggtggctggggacagcggatttgctgcttactcccggtatagaattggcaattacaaactgaacacagaccacagctcctctagtgataatatcgccctgctggtgcaggctagcgacaacgggccccagaaccagcggaatgcccctagaatcaccttcggcgggccatccgattctacaggcagcaaccagaatggagagaggtccggagctcgctctaagcagagacggccccagggcctgccaaacaataccgcctcctggtttactgctctgacccagcatgggaaggaagatctgaaattccccaggggacagggcgtgcctatcaacaccaattcaagcccagacgatcagattgggtactataggagggctacaaggagaatccggggaggcgacggaaagatgaaagatctgagccccagatggtacttttactatctgggaacaggaccagaggctggactgccttatggcgctaacaaggacggaatcatttgggtggccactgaaggcgctctgaatacccctaaagatcacattggaacccgcaacccagccaacaatgccgctatcgtgctgcagctgccacagggcaccacactgcccaagggcttctacgctgaggggagtaggggaggatcacaggcttcctctcgcagttcaagcaggtcccgcaattcctctcggaactctacaccagggagttcacggggaactagcccagctagaatggctggaaatggaggggacgccgctctggccctgctgctgctggatagactgaaccagctggagtctaagatgagtggcaaagggcagcagcagcagggacagacagtgactaagaaatccgccgctgaagcctctaagaaaccccggcagaagagaaccgctacaaaagcctacaatgtgacccaggcttttggcaggcgcggacctgagcagacacagggaaacttcggcgaccaggaactgattaggcagggcaccgattataagcactggccccagatcgctcagttcgcccctagtgcttcagccttctttggaatgtctcgcattggcatggaggtgacacctagtggcacttggctgacttacaccggggccatcaagctggacgataaagacccaaacttcaaggatcaggtcatcctgctgaacaagcatattgacgcctataaaaccttcccccctacagagcccaagaaagacaagaaaaagaaagccgatgaaacccaggctctgccccagaggcagaagaaacagcagacagtgactctgctgcctgccgctgatctggacgatttcagtaaacagctgcagcagagtatgagtagtgccgacagtacccaggcttaataatagtctaga

SEQ ID NO: 61 - DNA, 4040 bp, Artificial Sequence
  OC2.4 codon optimized DNA sequence
ggatccgcaccatggactggacttggattctgttcctggtcgccgccgccacccgagtgcatagcgcccgcgtgcagcccaccgagagcattgtgaggtttcccaacatcacaaatctgtgccctttcggcgaggtgtttaatgctactaggttcgccagcgtgtacgcttggaatcggaagagaatttctaactgcgtggccgactatagtgtgctgtacaacagcgcctccttctctacctttaagtgctatggggtgtctcccaccaaactgaatgacctgtgcttcacaaacgtgtacgccgacagttttgtgatccggggagatgaggtgagacagattgccccaggccagaccgggaagatcgctgactacaattataaactgcccgacgatttcacaggctgcgtgatcgcctggaacagtaacaatctggattcaaaagtgggcgggaactacaattatctgtacaggctgttccgcaagagcaacctgaaaccatttgagagggacatcagcactgaaatctaccaggccggctccaccccctgcaatggagtggagggcttcaactgttattttccactgcagagctacgggtttcagcccaccaacggggtgggatatcagccttaccgcgtggtggtgctgtccttcgaactgctgcacgctccagctaccgtgtgcggacccaagaaaagtactaacctggtgaagaacaagagggtgcagccaactgagtcaatcgtgcgctttccaaacattaccaatctgtgccccttcggggaagtgtttaatgccacacgcttcgcttccgtgtacgcctggaataggaagcgcatctcaaattgcgtggctgactatagcgtgctgtacaacagtgcttcattcagcacttttaagtgctatggcgtgtcacctactaaactgaatgacctgtgctttaccaacgtgtacgctgacagctttgtgattaggggggatgaggtgcgccagatcgctcctggacagactggaaagattgctgactataactacaaactgccagacgatttcaccggatgcgtgattgcctggaacagcaataagctggattccaaagtgggaggcaactataattacctgtatcggctgttcagaaagtctaacctgaaaccttttgagcgggacatctctaccgagatctaccaggccggaagtaccccatgcaatggcgtggaggggttcaactgctactttccactgcagtcttacggctttcagcccacctatggcgtggggtatcagccttacagagtggtggtgctgagcttcgaactgctgcatgctcctgctaccgtgtgcggaccaaagaaaagcaccaacctggtcaagaacaagcgggtgcagcctacagagtccatcgtgagatttcctaacattactaatctgtgcccattcggcgaagtgtttaatgctacaagatttgctagcgtgtacgcctggaatcggaagagaatctccaattgcgtggcagactattctgtgctgtacaactccgcttctttcagtacctttaagtgttacggagtgtccccaacaaaactgaatgacctgtgcttcacgaacgtgtacgccgactcttttgtgatccggggcgatgaggtgagacagatcgcccccggacagacaggcaatatcgctgactacaactataagctgcctgacgatttcactggatgcgtgattgcttggaattctaacaatctggatagtaaagtggggggaaattataactatctgtataggctgttccgcaagtctaacctgaaaccctttgagagggacatctcaaccgagatctaccaggctgggagcaccccttgcaatggggtgaagggattcaactgttactttccactgcagagctatggatttcagcccacatacggagtgggctaccagccatatagagtggtggtgctgagtttcgagctgctgcatgccccagctacagtgtgcggacctaagaaaagcacaaacctggtcaaaaacaagggaagcggagctactaacttcagcctgctgaagcaggctggagacgtggaggagaaccctggacctatggccgatagcaacgggactatcaccgtggaggaactgaagaaactgctggaacagtggaacctggtcatcggcttcctgtttctgacctggatctgtctgctgcagttcgcctatgctaacaggaatcgctttctgtacatcattaagctgattttcctgtggctgctgtggcctgtgactctggcctgcttcgtgctggccgccgtgtacaggatcaactggattaccggcgggatcgccattgctatggcctgtctggtgggcctgatgtggctgtcatactttatcgccagcttccgcctgtttgctcggaccagaagtatgtggtcattcaaccctgagacaaatattctgctgaacgtgccactgcacggcaccatcctgacacggcccctgctggagagcgaactggtcatcggcgccgtgattctgagggggcatctgcgcatcgctggccaccatctggggagatgcgacatcaaggatctgcccaaagaaattactgtggccacctctaggacactgagttactataagctgggagcttcccagagggtggctggcgacagcggatttgctgcttattccaggtaccgcatcgggaattacaaactgaacacagaccacagctcctctagtgataatattgccctgctggtgcaggctagcgacaacggaccccagaaccagcggaatgcccctagaatcaccttcggaggcccatcagatagcacaggctccaaccagaatggagagaggtcaggagctcgcagcaagcagagacggccccagggactgccaaacaataccgcctcttggtttactgctctgacccagcatggcaaggaagatctgaaattcccacggggccagggggtgcccattaacaccaattcaagccccgacgatcagatcggctactataggagggctacaaggagaattcggggaggagacgggaagatgaaagatctgagccctagatggtacttctactatctgggaacaggaccagaggctgggctgccttacggagctaacaaggacgggatcatttgggtggccactgaaggagctctgaatacccctaaagatcacatcgggaccagaaacccagccaacaatgccgctattgtgctgcagctgcctcagggaaccacactgccaaagggattctacgctgagggctcccggggaggcagccaggcttccagccggagctcaagccggagtagaaattcctctcggaactcaacaccaggcagttcaagggggacttccccagctaggatggctggaaatggaggcgacgccgctctggccctgctgctgctggatagactgaaccagctggagagcaagatgtccgggaaaggacagcagcagcagggacagacagtgactaagaaatccgccgctgaagcctctaagaaacctcggcagaagagaaccgctacaaaagcctataatgtgactcaggcttttggaaggcgcggaccagagcagacccagggaaacttcggcgaccaggaactgatcaggcagggcaccgattacaagcactggccccagattgctcagttcgccccttccgcttctgccttctttgggatgtcccgcatcggaatggaggtgacaccatctggaacttggctgacttataccggcgccattaagctggacgataaagaccccaacttcaaggatcaggtcatcctgctgaacaagcatattgacgcctacaaaaccttcccccctacagagcccaagaaagacaagaaaaagaaagccgatgaaacccaggctctgccccagagacagaagaaacagcagacagtgactctgctgcctgccgctgatctggacgatttctcaaaacagctgcagcagagcatgtcaagtgccgattcaactcaggcttaatagtctaga

SEQ ID NO: 62 - DNA, 2720 bp, Artificial Sequence
  SVF-14 codon optimized DNA sequence
ggatccgcaccatggattggacttggattctgtttctggtcgccgccgccacacgagtgcattccgcccgagtgcagcctaccgagagtattgtccgattccctaacattactaatctgtgcccattcggcgaggtgtttaacgccaccaggtttgctagcgtgtacgcctggaaccggaagagaatcagcaattgcgtggccgactactccgtgctgtataattccgcttctttcagtacctttaagtgctacggcgtgtcacccactaaactgaacgacctgtgcttcaccaacgtgtacgccgacagctttgtgatccggggggatgaagtgagacagattgcccctggccagacagggaagatcgctgactacaactataaactgccagacgatttcactggctgcgtgatcgcctggaattctaacaatctggatagtaaagtgggcgggaactacaattatctgtacaggctgttccgcaagagcaacctgaaaccatttgagcgggacatctccacagaaatctaccaggccggatctactccctgcaacggagtggagggcttcaattgttactttcctctgcagagctatggcttccagccaaccaatggcgtggggtaccagccctatagagtggtggtgctgagctttgaactgctgcacgctccagctaccgtgtgcggacccaagaaatctacaaacctggtgaagaacaagcgggtgcagcccacagagagtatcgtgagattccccaatatcactaatctgtgccctttcggcgaagtgtttaacgctacacgctttgcctccgtgtacgcttggaacaggaagcgcatttctaactgcgtggctgactacagtgtgctgtataattcagctagcttctccacttttaagtgctacggagtgtcccctaccaaactgaacgacctgtgctttactaacgtgtacgctgactcttttgtgattaggggcgatgaggtgcgccagatcgctccaggacagaccggaaagattgctgactataactacaaactgcctgacgatttcacaggatgcgtgattgcctggaattcaaacaatctggatagcaaagtgggaggcaactataattacctgtatcggctgttcagaaagagcaacctgaaaccttttgagagggacatcagtaccgagatctaccaggccgggtcaactccatgcaacggcgtggaggggttcaattgctatttcccactgcagagctatggattccagccaaccaacggagtgggctaccagccctatcgcgtggtggtgctgagttttgaactgctgcatgctcctgctaccgtgtgcggaccaaagaaatctaccaacctggtcaaaaacaagggaagcggagctactaacttcagcctgctgaagcaggctggagacgtggaggagaaccctggacctatggcttccgataatggcccacagaaccagcggaatgctcccagaatcacttttgggggaccttctgacagtaccggctctaaccagaatggcgagaggagtggagctcgctcaaagcagagacggccccaggggctgccaaacaataccgcctcctggttcacagctctgactcagcacggaaaggaagatctgaaatttccccggggccagggggtgcctatcaacactaatagctcccctgacgatcagattggatactataggagggctaccaggagaatcaggggaggggacggcaagatgaaagatctgtctccacgctggtacttctactatctgggaaccggaccagaggctggactgccttatggagctaacaaggacggcatcatttgggtggccacagaaggggctctgaacactcccaaagatcatattggcacaagaaatcctgccaacaatgccgctatcgtgctgcagctgccacaggggaccacactgcccaagggcttttacgctgaggggagcaggggaggctcccaggcttctagtcgctcaagctcccggtcaagaaactctagtaggaatagcacccctgggtcaagccggggaacaagcccagccagaatggctggaaacggaggagacgccgctctggccctgctgctgctggatcgcctgaatcagctggagtccaagatgtctgggaaaggacagcagcagcagggacagactgtgaccaagaaatccgccgctgaagcctctaagaaaccaaggcagaagcgcacagccactaaagcttacaacgtgacccaggctttcggaaggcgcggaccagagcagacacaggggaattttggcgaccaggaactgattcggcagggcaccgattataagcactggccacagatcgcccagttcgctccctcagccagcgccttcttcggaatgagcagaattggcatggaggtgaccccctccgggacatggctgacctacacaggagccatcaagctggacgataaagaccctaacttcaaagatcaggtcatcctgctgaacaagcatattgacgcctataaaacttttccccctaccgagcccaagaaagacaagaaaaagaaagccgatgaaacacaggctctgccacagcggcagaagaaacagcagactgtgaccctgctgcccgccgctgatctggacgatttcagtaaacagctgcagcagtcaatgtcttccgccgattcaactcaggcataatagtctaga

SEQ ID NO: 63 - DNA, 5222 bp, Artificial Sequence
  SVF-15 codon optimized DNA sequence
ggatccgcaccatggactggacatggattctgtttctggtcgctgctgctacccgagtgcattctgccttcgtctttctggtgctgctgcctctggtcagctcccagtgcgtgaacctgaccacaaggacacagctgccccctgcctatacaaattccttcactcggggcgtgtactatcctgacaaagtgtttagatctagtgtgctgcactccacacaggatctgtttctgccattcttttctaacgtgacttggttccacgccatccacgtgagcgggaccaatggaacaaagaggttcgacaatccagtgctgccctttaacgatggcgtgtacttcgccagtaccgagaagtcaaacatcattcgcgggtggatctttggaactaccctggacagcaaaacccagtccctgctgatcgtgaacaatgccacaaacgtggtcatcaaggtgtgcgaattccagttttgtaatgatcccttcctgggcgtgtactatcataagaacaacaagtcttggatggagagtgaatttcgcgtgtattcaagcgccaacaattgcacatttgagtacgtgagccagcctttcctgatggacctggaaggcaagcaggggaatttcaaaaacctgcgggagttcgtgtttaagaatattgatggctacttcaagatctactctaaacacacccccatcaacctggtgcgggacctgcctcaggggttcagtgccctggagcctctggtggatctgccaatcggaattaacatcacccggtttcagacactgctggccctgcatagatcctacctgacaccaggcgactcctctagtggctggactgctggggccgctgcctactatgtggggtatctgcagccccggaccttcctgctgaaatacaacgagaatggaactattaccgacgctgtggattgcgccctggaccccctgtccgaaactaagtgtaccctgaaatcttttaccgtggagaagggaatctatcagacaagcaatttcagggtgcagccaactgaatccattgtgcgctttccaaatatcaccaacctgtgcccctttggcgaggtgttcaacgctacaagattcgccagcgtgtacgcttggaataggaagcgcatcagcaactgcgtggccgactattccgtgctgtacaactccgcttctttcagtacctttaagtgctatggagtgtcacccaccaaactgaatgacctgtgctttacaaacgtgtacgccgattccttcgtgattaggggcgacgaggtgcgccagatcgctcctggacagaccggcaagattgctgactacaattataaactgccagacgatttcacaggctgcgtgatcgcctggaactctaacaatctggatagtaaagtgggcgggaactacaattatctgtacaggctgtttcgcaagagcaatctgaaacccttcgagcgggacattagtactgaaatctaccaggccggctcaaccccttgcaatggggtggagggcttcaactgttatttcccactgcagtcctacgggttccagcccacaaacggagtgggctatcagccttacagagtggtggtgctgtcttttgaactgctgcacgctccagctacagtgtgcggacccaagaaaagtactaatctggtgaagaacaaatgcgtgaacttcaacttcaacggactgacagggactggagtgctgaccgagagcaacaagaaattcctgccatttcagcagttcggaagggacatcgccgatacaactgacgctgtgcgcgacccccagaccctggaaattctggatatcacaccttgctcattcggaggcgtgagcgtgattacccccggcaccaatacatccaaccaggtggccgtgctgtatcaggacgtgaattgtacagaggtgccagtggctatccacgccgatcagctgactcccacctggcgggtgtacagcactggctccaacgtgtttcagaccagagccggatgcctgattggcgctgagcatgtgaacaatagttatgaatgcgacattccaatcggcgccgggatctgtgcttcataccagacacagactaactctccccggagagccaggagtgtggcttcacagagcatcattgcctataccatgtcactgggggccgaaaatagcgtggcttactctaacaatagtattgctatccccacaaacttcactatttctgtgaccacagagatcctgcccgtgagcatgaccaagacatctgtggactgcaccatgtatatttgtggcgattcaacagaatgcagcaacctgctgctgcagtacggaagcttttgtacacagctgaatcgcgccctgactggcatcgctgtggagcaggacaaaaacactcaggaagtgttcgcccaggtgaagcagatctacaaaaccccacccatcaaggactttgggggcttcaacttcagccagattctgcctgatccatcaaagcccagcaaaaggtcctttatcgaggacctgctgttcaacaaggtgaccctggctgatgccggcttcatcaaacagtatggggattgcctgggagacattgctgcccgcgacctgatctgtgcccagaagtttaatgggctgaccgtgctgcctccactgctgacagatgaaatgattgcccagtacacatctgctctgctggccgggactatcaccagtggatggactttcggagccggcgctgccctgcagattccttttgccatgcagatggcttataggttcaacgggatcggagtgacccagaatgtgctgtacgagaaccagaagctgatcgccaatcagtttaacagcgccattggcaaaatccaggactccctgtcaagcactgcttctgccctggggaaactgcaggatgtggtgaatcagaacgctcaggccctgaataccctggtgaagcagctgtcctctaacttcggggccattagttcagtgctgaatgatatcctgtccaggctggacccccctgaggctgaagtgcagattgaccggctgatcacaggaagactgcagagcctgcagacctacgtgacacagcagctgattagggctgccgaaatccgcgcttccgccaatctggctgccaccaagatgtctgagtgcgtgctggggcagagtaagcgcgtggacttttgtggcaaagggtatcacctgatgtccttcccccagtctgcccctcacggagtggtgtttctgcatgtgacctacgtgcctgctcaggagaagaacttcactaccgctccagccatctgccacgatggcaaagcccattttccccgggaaggcgtgttcgtgtccaacgggacccattggtttgtgacacagagaaatttctacgagcctcagatcattacaactgacaataccttcgtgagcggaaactgtgacgtggtcatcggcatcgtgaacaataccgtgtacgatcctctgcagccagagctggacagctttaaggaggaactggataagtacttcaaaaatcacacctcccccgacgtggatctgggcgacattagtgggatcaatgcctcagtggtgaacattcagaaggagatcgacagactgaacgaagtggctaaaaatctgaacgagagcctgattgatctgcaggagctgggcaagtatgaacagtacatcaaatggccttggtatatttggctgggattcatcgccggcctgattgctatcgtgatggtgactatcatgctgtgctgtatgacctcttgctgtagttgcctgaagggctgctgttcatgtgggagctgctgtaaattcgacgaggacgattccgaaccagtgctgaagggcgtgaaactgcactacactggaagcggagctactaacttcagcctgctgaagcaggctggagacgtggaggagaaccctggacctatggcctctgataacggccctcagaatcagaggaacgctccacgcatcactttcggcggaccatccgactctaccggcagcaatcagaacggggagcggagtggagccagatcaaagcagaggcgcccacagggcctgcccaacaataccgccagctggttcactgctctgacccagcatgggaaggaagatctgaaatttcctcgggggcagggagtgccaatcaatactaacagctccccagacgatcagattggatactataggagagctaccaggcgcatcaggggaggcgatggcaagatgaaagacctgtccccccgctggtatttctactatctgggaacaggaccagaagctggcctgccttacggggctaacaaagacggcatcatttgggtggccacagagggggctctgaacactcctaaggatcacattggaaccagaaatccagccaacaatgctgctatcgtgctgcagctgccacagggaaccacactgcctaagggattctacgctgagggcagcaggggaggcagccaggcttctagtcgctcaagctcccggtcaagaaattctagtcggaacagcacaccaggatcaagccggggcactagcccagctagaatggctggaaatggaggggacgctgccctggccctgctgctgctggatagactgaaccagctggaatccaagatgtctggaaaaggccagcagcagcaggggcagacagtgaccaagaagagcgccgctgaggcttccaagaaacctaggcagaagagaaccgctacaaaagcctataatgtgactcaggccttcggccggagaggaccagagcagacccagggaaactttggcgaccaggaactgattaggcagggcaccgattacaagcactggcctcagatcgctcagttcgccccaagtgcttcagccttctttgggatgtcacgcattggaatggaagtgactcccagcgggacctggctgacttataccggagccatcaagctggacgataaagatcctaacttcaaggaccaggtcatcctgctgaacaagcatatcgacgcctacaaaacatttccacccactgaacccaagaaagataagaaaaagaaagccgacgagacacaggctctgccacagcggcagaagaaacagcagacagtgactctgctgcccgctgccgatctggacgatttctcaaaacagctgcagcagtcaatgtcttccgccgattcaactcaggcataatagtctaga

SEQ ID NO: 64 - protein, 1120 aa, Artificial Sequence
  OC2.3 (N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 65 - protein, 1120 aa, Artificial Sequence
  OC2.3 (N439K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 66 - protein, 1120 aa, Artificial Sequence
  OC2.3 (K417N, E484K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 67 - protein, 1120 aa, Artificial Sequence
  OC2.3 (K417N, N439K, E484K, N501Y) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 68 - protein, 1339 aa, Artificial Sequence
  OC2.4 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 69 - protein, 899 aa, Artificial Sequence
  SVF-14 polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 70 - protein, 1733 aa, Artificial Sequence
  SVF-15 polypeptide sequence
MDWTWILFLVAAATRVHSAFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGSGATNFSLLKQAGDVEENPGPMASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 71 - DNA, 3842 bp, Artificial Sequence
  SVF-13 (OC-13) codon optimized DNA sequence (S protein)
ggatccgcaccatgtttgtctttctggtcctgctgcccctggtctcatcacagtgcgtcaacctgactacacgaacccagctgccacctgcttatacaaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgattctgagcccgtgctgaagggagtgaagctgcattacacctaatagtctaga

SEQ ID NO: 72 - protein, 1273 aa, Artificial Sequence
  SVF-13 (OC-13) polypeptide sequence (S protein)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 73 - DNA, 1346 bp, Artificial Sequence
  SVF-10.2 (OC-10.2) codon optimized DNA sequence
ggatccgcaccatggcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagtaataatagtctaga

SEQ ID NO: 74 - protein, 440 aa, Artificial Sequence
  SVF-10.2 (OC-10.2) polypeptide sequence
MARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 75 - DNA, 1397 bp, Artificial Sequence
  SVF-10.3 (OC-10.3) codon optimized DNA sequence
ggatccgcaccatggactggacctggattctgttcctggtggccgccgccacacgggtgcacagcgcccgagtccagcctactgagtctattgtccgctttcctaacatcactaatctgtgcccttttggcgaggtcttcaacgctacacggtttgcttccgtgtacgcctggaatcggaagagaatctctaactgcgtggccgactacagtgtgctgtataacagcgcctccttctctacctttaagtgctacggcgtgtcccccaccaaactgaatgacctgtgcttcacaaacgtgtatgccgactcttttgtgatccggggggatgaggtgagacagattgccccaggacagactggcaagatcgctgactacaattataaactgcccgacgatttcaccggctgcgtgatcgcctggaacagcaacaatctggattccaaagtgggcgggaactacaattatctgtaccggctgttcagaaagtccaatctgaaaccctttgagagggacatcagtactgaaatctaccaggccgggtcaaccccttgcaatggggtggagggattcaactgttactttccactgcagagttatggatttcagcccaccaacggagtgggctaccagccttatcgcgtggtggtgctgtctttcgaactgctgcacgctccagctacagtgtgcggacccaagaaatcaactaacctggtgaagaacaagcgggtgcagcctactgagagcatcgtgagatttcctaacattaccaatctgtgcccattcggcgaagtgtttaatgctacaagattcgccagcgtgtacgcttggaataggaagcgcatctcaaattgcgtggctgactacagcgtgctgtataacagtgcttcattcagcacttttaagtgctacggagtgagtccaactaaactgaatgacctgtgctttaccaacgtgtatgctgactcatttgtgattaggggcgatgaggtgcgccagatcgctcctggccagacagggaagattgctgactataattacaaactgccagacgatttcactggatgcgtgattgcctggaactctaacaatctggatagtaaagtgggaggcaactataattacctgtataggctgttccgcaagtctaatctgaaaccatttgagcgggacatcagcaccgagatctaccaggccggctccaccccctgcaatggagtggagggcttcaactgctattttccactgcagagctatgggtttcagcccacaaacggggtgggataccagccttatagagtggtggtgctgtccttcgaactgctgcatgcccctgctacagtgtgcggcccaaagaaatctaccaacctggtcaagaacaagtaataatagtctaga

SEQ ID NO: 76 - protein, 457 aa, Artificial Sequence
  SVF-10.3 (OC-10.3) polypeptide sequence
MDWTWILFLVAAATRVHSARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

(19) United States
(12) Patent Application Publication
(10) Pub. No.: US 2023/0330211 A1
(43) Pub. Date: Oct. 19, 2023

(54) COMPOSITIONS AND METHODS FOR TREATING AND PREVENTING CORONAVIRUSES

(71) Applicant: Svenska Vaccinfabriken Produktion AB, Sollentuna (SE)

(72) Inventors: Matti Sällberg, Sollentuna (SE); Lars Frelin, Sollentuna (SE)

(21) Appl. No.: 17/913,751

(22) PCT Filed: Mar. 24, 2021
(86) PCT No.: PCT/US2021/023991

§ 371 Date: Sep. 22, 2022

Related U.S. Application Data
(60) Provisional application No. 63/000,978, filed on Mar. 27, 2020.
(60) Provisional application No. 63/088,228, filed on Oct. 6, 2020.
(60) Provisional application No. 63/141,875, filed on Jan. 26, 2021.
(60) Provisional application No. 63/156,660, filed on Mar. 4, 2021.

(51) Int. Cl. A61K 39/215; A61P 37/04

(52) U.S. Cl. CPC A61K 39/215; A61P 37/04; A61K 2039/53

(57) ABSTRACT

Disclosed herein are immunogenic compositions or product combinations of engineered SARS-CoV nucleic acids, genes, peptides, or proteins that can be used to elicit an immune response against a SARS-CoV infection or infection by another coronavirus, including SARS-CoV-2 and variants thereof. Also disclosed are methods of using the immunogenic compositions or product combinations in subjects to generate immune responses and neutralizing antibodies against SARS-CoV or another coronavirus by administering the compositions or combinations with a nucleic acid prime and polypeptide boost approach.

### CROSS-REFERENCE TO RELATED APPLICATIONS

This application claims the benefit of priority of U.S. Provisional Patent Application No. 63/000,978, filed Mar. 27, 2020, U.S. Provisional Patent Application No. 63/088,228, filed Oct. 6, 2020, U.S. Provisional Patent Application No. 63/141,875, filed Jan. 26, 2021, and U.S. Provisional Patent Application No. 63/156,660, filed Mar. 4, 2021, each of which is hereby expressly incorporated by reference in its entirety, including any appendices filed therewith.

### REFERENCE TO SEQUENCE LISTING

The present application is being filed along with a Sequence Listing in electronic format. The Sequence Listing is provided in a file entitled SeqListingSVF006WO.TXT, which was created on Mar. 24, 2021 and is 368,069 bytes in size. The information in the electronic Sequence Listing is hereby expressly incorporated by reference in its entirety.

### FIELD

Aspects of the present disclosure relate generally to immunogenic compositions or product combinations of engineered SARS-CoV-2 nucleic acids, genes, peptides, or proteins that can be used to elicit an immune response against a SARS-CoV-2 infection or infection by another coronavirus. This immune response includes activation of cytotoxic immune cells and immune cells that produce neutralizing antibodies against SARS-CoV-2 or another coronavirus, including variants thereof. The disclosure also relates generally to methods of using or administering the immunogenic compositions or product combinations described herein to subjects to generate immune responses including but not limited to the production of neutralizing antibodies against SARS-CoV-2 or another coronavirus, for example by administering the compositions or combinations with a homologous or heterologous nucleic acid and/or polypeptide prime and nucleic acid and/or polypeptide boost approach.

### BACKGROUND

The 2019 coronavirus pandemic caused by the SARS-CoV-2 (2019-nCoV) virus has resulted in devastating losses of human life, impact on the global economy, and pressure on the public health infrastructure around the world. Although human coronavirus immunotherapies or vaccines directed to the SARS-CoV-2 virus are beginning to be approved, long-term efficacy and safety profiles have not been performed. Furthermore, additional variants or mutants of the SARS-CoV-2 virus, some of which have been shown to be more contagious or virulent than the originally identified strain, are emerging. As such, there is a great need for new treatments and prophylaxes against SARS-CoV-2 and other coronaviruses.

### SUMMARY

Speed in therapeutic and vaccine development against SARS-CoV-2 and other potential new coronavirus strains or mutants is of utmost importance. Genetic analysis of the virus shows that the most variable components of SARS-CoV-2 and coronaviruses in general is the spike (S) protein, which includes the receptor binding domains (RBD). The RBD of SARS-CoV-2 has approximately 75% homology with the SARS virus of 2003 (SARS-CoV-1) and other coronaviruses (Wu A et al. “Genome Compositions and Divergence of the Novel Coronavirus (2019-nCoV) Originating in China” Cell Host Microbe. (2020); 27(3):325-328). This suggests that existing immunotherapies and vaccine candidates against other coronaviruses such as SARS-CoV-1 will not be useful in protecting against SARS-CoV-2.

Disclosed herein are unique candidates for use as immunogenic compositions against SARS-CoV-2 that allow for rapid validation and large-scale production. Described herein is the use of a heterologous prime-boost immunization approach using a nucleic acid (DNA or RNA) prime and a polypeptide boost administration schedule. A nucleic acid prime allows for detection of neutralizing antibodies within one or two weeks from a single dose. This is due to better T cell priming, as compared to a protein/adjuvant mix.

In some embodiments, the immunogenic compositions or product compositions described herein are nucleic acids and/or polypeptides. In some embodiments, the nucleic acids are DNA or RNA. In some embodiments, the immunogenic compositions or product compositions are intended to be administered to an animal, such as a mammal, mouse, rabbit, cat, dog, primate, monkey, or human, to induce an immunogenic response against the SARS-CoV-2 virus or other coronavirus. In some embodiments, the immunogenic response comprises, consists essentially of, or consist of formation of active immune cells, such as cytotoxic T cells or immune cells that produce inactivating antibodies against the SARS-CoV-2 virus, other coronavirus, or any antigen, polypeptide, protein, nucleic acid, or genome component of the virus. In some embodiments, the immunogenic compositions or product compositions are intended to be administered to an animal, such as a mammal, mouse, rabbit, cat, dog, primate, monkey, or human, to generate neutralizing antibodies against the SARS-CoV-2 virus or other coronavirus in the animal. In some embodiments, the immunogenic compositions or product compositions are administered to individuals that are at risk of contracting SARS-CoV-2 or are not currently infected with SARS-CoV-2. In some embodiments, the immunogenic compositions or product combinations provide lasting immunogenic protection against a SARS-CoV-2 infection.

Some alternatives described herein concern nucleic acids comprising, consisting essentially of, or consisting of at least one SARS-CoV-2 nucleic acid component, preferably joined with a nucleic acid encoding an IgE leader sequence (e.g., a nucleic acid encoding the amino acid sequence MDWTWILFLVAAATRVHS (SEQ ID NO: 44), or an IgE leader nucleic acid sequence having at least 90%, 91%, 92%, 93%, 94%, 95%, 96%, 97%, 98%, 99%, or 100% homology or sequence identity to SEQ ID NO: 43, as well as, use of such nucleic acids and/or the proteins encoded thereby as a medicament, including medicaments that treat or inhibit SARS-CoV-2 infection.

In some alternatives, the at least one SARS-CoV-2 nucleic acid component comprises, consists essentially of, or consists of an S protein sequence, RBD sequence, M protein sequence, NP protein sequence, E protein sequence, or HE protein sequence. In some alternatives, the at least one SARS-CoV-2 nucleic acid component is found as the wild-type sequence. Some alternatives concern nucleic acids and the use thereof, wherein the nucleic acids share or comprise at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 1-12 or an amount of sequence identity to any one or more of SEQ ID NO: 1-12 that is within a range defined by any two of the aforementioned percentages. In some alternatives, the at least one SARS-CoV-2 nucleic acid component contemplated for inclusion in the compositions and the uses described herein are human codon optimized sequences of the aforementioned wild-type sequences. In some alternatives, for example, the nucleic acids share or comprise 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 13-24, 39-40, 57-63, 71, 73, or 75, or an amount of sequence identity to any one or more of SEQ ID NO: 13-24, 39-40, 57-63, 71, 73, or 75 that is within a range defined by any two of the aforementioned percentages. In some alternatives, the nucleic acids referenced above are used for the prevention, treatment or inhibition of a SARS-CoV-2 infection in a subject, such as a mammal, preferably a human. Accordingly, some alternatives include the use of a nucleic acid having at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 1-24, 39-40, 57-63, 71, 73, or 75, or an amount of sequence identity to any one or more of SEQ ID NO: 1-24, 39-40, 57-63, 71, 73, or 75, that is within a range defined by any two of the aforementioned percentages as a medicament, such as for the prevention, treatment, amelioration, or inhibition of a SARS-CoV-2 infection in a subject, such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both.

Some alternatives provided herein concern polypeptides comprising, consisting essentially of, or consisting of at least one SARS-CoV-2 polypeptide component. In some alternatives, the at least one SARS-CoV-2 polypeptide component comprises, consists essentially of, or consists of an S protein sequence, RBD sequence, M protein sequence, NP protein sequence, E protein sequence, or HE protein sequence. In some embodiments, the polypeptides, which may be provided in a composition or method described herein share or comprise at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 25-36, 41-42, 64-70, 72, 74, or 76, or an amount of sequence identity to any one or more of SEQ ID NO: 25-36, 41-42, 64-70, 72, 74, or 76, that is within a range defined by any two of the aforementioned percentages. In some alternatives, the polypeptides are used as a medicament, such as for the prevention, treatment or inhibition of SARS-CoV-2 in a subject such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both. In some embodiments, the polypeptides are translated from the wild-type or codon optimized sequences referenced above. In some embodiments, the polypeptides are recombinantly expressed. In some embodiments, the polypeptides are recombinantly expressed in a mammalian, bacterial, yeast, insect, or cell-free system. Accordingly, some alternatives include the use of a polypeptide having at least 50%, 60%, 70%, 80%, 85%, 86%, 87%, 88%, 89%, 90%, 91%, 92%, 93%, 94%, 95%, 96%, 97%, 98%, 99%, or 100% homology or sequence identity to any one or more of SEQ ID NO: 25-36, 4142, 64-70, 72, 74, or 76, or an amount of sequence identity to any one or more of SEQ ID NO: 25-36, 4142, 64-70, 72, 74, or 76, that is within a range defined by any two of the aforementioned percentages as a medicament, such as for the prevention, treatment, amelioration, or inhibition of a SARS-CoV-2 infection in a subject, such as a mammal, preferably a human, which may, optionally, be selected or identified to receive a medicament for the prevention, treatment, amelioration, or inhibition of a SARS-CoV-2 infection.

In some alternatives, the nucleic acids or polypeptides also comprise at least one autocatalytic peptide cleavage site. In some alternatives, the at least one autocatalytic peptide cleavage site is a P2A autocatalytic peptide cleavage site. In some alternatives, the at least one SARS-CoV-2 nucleic acid component or the at least one SARS-CoV-2 polypeptide component are separated by the at least one autocatalytic peptide cleavage site.

In some alternatives, at least one HDAg strain sequence is provided in the nucleic acids or polypeptides referenced above, such as 1, 2, 3, 4, 5, 6, 7, 8, 9, or 10 HDAg strain sequences selected from HDAg genotype 1A, HDAg genotype 1B, HDAg genotype 2A, or HDAg genotype 2B or any combination thereof. In some alternatives, four HDAg strain sequences are provided in the nucleic acids or polypeptides referenced thereof. In some alternatives, the four HDAg strain sequences comprise one copy each of HDAg genotype 1A, HDAg genotype 1B, HDAg genotype 2A, and HDAg genotype 2B. In some alternatives, there are less than four HDAg strain sequences in the nucleic acids or polypeptides. In some alternatives, the HDAg strain sequences are found in tandem in the nucleic acids or polypeptides. In some alternatives, the HDAg strain sequences are separated by autocatalytic peptide cleavage sites. In other alternatives, the HDAg strain sequences are found in tandem with no linker, a linker of at least 1 nucleotide or amino acid, or without an autocatalytic peptide cleavage site in between. In some alternatives, the SARS-CoV-2 or other coronavirus sequences are found either upstream or downstream of the HDAg strain sequences. In some alternatives, the SARS-CoV-2 or other coronavirus sequences are separated from the HDAg strain sequences with an autocatalytic peptide cleavage site. In some alternatives, the autocatalytic peptide cleavage site is a P2A autocatalytic peptide cleavage site. In some alternatives, the constructs SVF-8 (OC-8) and SVF-9 (OC-9) comprise, consist essentially of, or consist of HDAg strain sequences.

In some alternatives, the immunogenic compositions or product compositions comprise, consist essentially of, or consist of a nucleic acid, described above (e.g., any one or more of SEQ ID NO: 1-24, 39-40, 57-63, 71, 73, or 75), and a polypeptide, described above (e.g., any one or more of SEQ ID NO: 25-36, 41-42, 64-70, 72, 74, or 76). In some alternatives, the immunogenic compositions or product compositions are administered to a subject in a heterologous prime-boost approach. In some alternatives, the prime dose comprises the nucleic acid and the boost dose comprises the polypeptide. In some alternatives, the prime dose comprises any one or more of the aforementioned polypeptides and the boost dose comprises any one or more of the aforementioned nucleic acids. In some alternatives, the immunogenic compositions or product compositions are administered to a subject as a homologous prime-boost approach. In some alternatives, the prime dose comprises any one or more of the aforementioned nucleic acids and the boost dose comprises either the same nucleic acid or a different nucleic acid. In some alternatives, the prime dose comprises any one or more of the aforementioned polypeptides and the boost dose comprises either the same polypeptide or a different polypeptide. In some alternatives, the immunogenic compositions or product compositions further comprise an adjuvant. In some embodiments, the adjuvant is alum and/or QS21. In some alternatives, the nucleic acid is provided as a recombinant vector. In some alternatives, the recombinant vector is pVAX1. In some alternatives, the immunogenic compositions or product compositions are used for the prevention, treatment or inhibition of SARS-CoV-2 in a subject, such as a mammal, preferably a human, which may, optionally, be selected or identified to receive a medicament for the prevention, treatment, amelioration, or inhibition of a SARS-CoV-2 infection. Such subjects can be selected or identified by clinical evaluation or diagnostic evaluation or both.

Some alternatives described herein concern methods of generating an immune response in a subject, preferably a human, using the immunogenic compositions, product compositions, nucleic acids, or polypeptides described above (e.g., any one or more of SEQ ID NO: 1-36, 39-42, or 57-70). In some alternatives, the methods comprise a heterologous prime-boost approach. In some alternatives, at least one prime dose is administered to the subject and at least one boost dose is administered to the subject. In some alternatives, the at least one prime dose is a nucleic acid. In some alternatives, the at least one boost dose is a polypeptide. In some alternatives, the at least one boost dose comprises an adjuvant, such as alum and/or QS21. In some alternatives, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points. In some alternatives, the methods comprise a homologous prime-boost approach. In some alternatives, the method further comprises administration of an antiviral therapy, such as dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, AT-100, or TJM2, or a stem cell therapy, or any combination thereof.

Additional alternatives concern an injection device comprising any one or more of the compositions described herein, such as any one or more of the nucleic acids or polypeptides set forth in any one or more of SEQ ID NO: 1-36, 39-42, or 57-70. Such injection devices can comprise a single dose of such nucleic acid or polypeptide and such injection devices can have modified needle designs configured to enhance delivery of the nucleic acid or polypeptide or both. Such injection devices can be used with or without electroporation. Contemplated injection devices, which can include any one or more of the nucleic acids or polypeptides of SEQ ID NO: 1-36, 39-42, or 57-70 are described in U.S. Pat. App. Pub. No. 2016/0235928; PCT App. Pub. No. WO2014064534; U.S. Pat. Nos. 6,610,044; 6,132,419; 6,379,966; 6,897,068; 7,015,040; 7,214,369; 7,473,419; and 7,589,059, all of which are hereby expressly incorporated by reference in their entireties.

Some aspects of the present invention are related to the following numbered alternatives:

1. A nucleic acid comprising at least one nucleic acid sequence encoding a SARS-CoV-2 polypeptide and at least one nucleic acid sequence encoding a P2A autocatalytic polypeptide cleavage site.

2. The nucleic acid of alternative 1, wherein the at least one nucleic acid sequence encoding a SARS-CoV-2 polypeptide comprises a nucleic acid sequence encoding an RBD polypeptide and a nucleic acid sequence encoding an NP polypeptide.

3. The nucleic acid of alternative 1 or 2, wherein the nucleic acid shares or comprises at least 90%, 91%, 92%, 93%, 94%, 95%, 96%, 97%, 98%, 99%, or 100% homology or sequence identity to SEQ ID NO: 1 or 13.

4. The nucleic acid of alternative 1, wherein the at least one nucleic acid sequence encoding a SARS-CoV-2 polypeptide comprises a nucleic acid sequence encoding an RBD polypeptide, a nucleic acid sequence encoding an M polypeptide, and a nucleic acid sequence encoding an NP polypeptide.

5. The nucleic acid of any one of alternatives 1-2 or 4, wherein the nucleic acid shares or comprises at least 90%, 91%, 92%, 93%, 94%, 95%, 96%, 97%, 98%, 99%, or 100% homology or sequence identity to any one or more of SEQ ID NO: 2-3, 14, or 15.

6. The nucleic acid of alternative 4, wherein the RBD polypeptide is an RBD tandem repeat single chain dimer polypeptide.

7. The nucleic acid of alternative 6, wherein the RBD tandem repeat single chain dimer polypeptide comprises a K417N, N439K, E484K, or N501Y mutation with reference to the full S protein (e.g., as set forth in NCBI Accession No. YP_009724390), or any combination thereof.

8. The nucleic acid of alternative 6 or 7, wherein the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50.

9. The nucleic acid of any one of alternatives 6-8, wherein the nucleic acid shares or comprises at least 90%, 91%, 92%, 93%, 94%, 95%, 96%, 97%, 98%, 99%, or 100% homology or sequence identity to SEQ ID NO: 39.

10. The nucleic acid of any one of alternatives 1-2 or 4, further comprising a 5′ IgE leader nucleic acid sequence.

11. The nucleic acid of alternative 10, wherein the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43.

12. The nucleic acid of alternative 10 or 11, wherein the RBD polypeptide is an RBD tandem repeat single chain dimer polypeptide.

13. The nucleic acid of alternative 12, wherein the RBD tandem repeat single chain dimer polypeptide comprises a K417N, N439K, E484K, or N501Y mutation with reference to the full S protein (e.g., as set forth in NCBI Accession No. YP_009724390), or any combination thereof.

14. The nucleic acid of alternative 12 or 13, wherein the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50.

15. The nucleic acid of any one of alternatives 10-14, wherein the nucleic acid shares or comprises at least 90%, 91%, 92%, 93%, 94%, 95%, 96%, 97%, 98%, 99%, or 100% homology or sequence identity to any one or more of SEQ ID NO: 40, 57-60, or 62.

16. The nucleic acid of alternative 10 or 11, wherein the RBD polypeptide comprises three tandem copies of RBD.

17. The nucleic acid of alternative 16, wherein the three tandem copies of RBD each comprise a K417N, N439K, E484K, or N501Y mutation with reference to the full S protein (e.g., as set forth in NCBI Accession No. YP_009724390), or any combination thereof, or none of these mutations.

18. The nucleic acid of alternative 16 or 17, wherein the nucleic acid shares or comprises at least 90%, 91%, 92%, 93%, 94%, 95%, 96%, 97%, 98%, 99%, or 100% homology or sequence identity to SEQ ID NO: 61.

19. The nucleic acid of alternative 1, wherein the at least one nucleic acid sequence encoding a SARS-CoV-2 polypeptide comprises a nucleic acid sequence encoding an RBD polypeptide and a nucleic acid sequence encoding an M polypeptide.

20. The nucleic acid of alternative 1 or 9, wherein the nucleic acid shares or comprises at least 90%, 91%, 92%, 93%, 94%, 95%, 96%, 97%, 98%, 99%, or 100% homology or sequence identity to SEQ ID NO: 4 or 16.

21. The nucleic acid of alternative 1, wherein the at least one nucleic acid sequence encoding a SARS-CoV-2 polypeptide comprises a nucleic acid sequence encoding a spike (S) polypeptide, a nucleic acid sequence encoding for a membrane (M) polypeptide, or a nucleic acid sequence encoding for an NP polypeptide, or any combination thereof.

22. The nucleic acid of alternative 21, wherein the S polypeptide comprises one or more mutations that improve expression, solubility, and/or immunogenicity.

23. The nucleic acid of alternative 21 or 22, wherein the S polypeptide comprises a K968P or V987P mutation with reference to the full S protein (e.g., as set forth in NCBI Accession No. YP_009724390), or both.

24. The nucleic acid of any one of alternatives 21-23, wherein the nucleic acid sequence encoding for the S polypeptide shares or comprises at least 90%, 91%, 92%, 93%, 94%, 95%, %%, 97%, 98%, 99%, or 100% homology or sequence identity to SEQ ID NO: 51.

25. The nucleic acid of any one of alternatives 21-24, further comprising a 5′ IgE leader nucleic acid sequence.

26. The nucleic acid of alternative 25, wherein the 5′ IgE leader nucleic acid sequence shares or comprises at least 900%, 91%, 92%, 93%, 94%, 95%, 96%, 97%, 98%, 99%, or 100% homology or sequence identity to SEQ ID NO: 43.

27. The nucleic acid of any one of alternatives 21-26, wherein the nucleic acid shares or comprises at least 90%, 91%, 92%, 93%, 94%, 95%, %%, 97%, 98%, 99%, or 100% homology or sequence identity to SEQ ID NO: 63.

28. A nucleic acid comprising at least one nucleic acid sequence encoding a SARS-CoV-2 polypeptide sharing or comprising at least 90, 91%, 92%, 93%, 94%, 95%, 96%, 97%, 98%, 99%, or 100% homology or sequence identity to any one or more of SEQ ID NO: 5-7, 17-19, 22-24, 73, or 75.

29. A nucleic acid comprising at least one nucleic acid sequence encoding a SARS-CoV-2 polypeptide and at least one nucleic acid sequence encoding a hepatitis D antigen (HDAg).

30. The nucleic acid of alternative 29, wherein the nucleic acid shares or comprises at least 90%, 91%, 92%, 93%, 94%, 95%, 96%, 97%, 98%, 99%, or 100% homology or sequence identity to SEQ ID NO: 8 or 20.

31. The nucleic acid of alternative 29, further comprising at least one nucleic acid sequence encoding a P2A autocatalytic polypeptide cleavage site.

32. The nucleic acid of alternative 29 or 31, wherein the nucleic acid shares or comprises at least 90%, 91%, 92%, 93%, 94%, 95%, 96%, 97%, 98%, 99%, or 100% homology or sequence identity to SEQ ID NO: 9 or 21.

33. A polypeptide comprising at least one SARS-CoV-2 polypeptide sequence and at least one P2A autocatalytic polypeptide cleavage site.

34. The polypeptide of alternative 33, wherein the at least one SARS-CoV-2 polypeptide sequence comprises an RBD polypeptide sequence and an NP polypeptide sequence.

35. The polypeptide of alternative 33 or 34, wherein the polypeptide shares or comprises at least 90%, 91%, 92%, 93%, 94%, 95%, 96%, 97%, 98%, 99%, or 100% homology or sequence identity to SEQ ID NO: 25.

36. The polypeptide of alternative 33, wherein the at least one SARS-CoV-2 polypeptide sequence comprises an RBD polypeptide sequence, an M polypeptide sequence, and an NP polypeptide sequence.

37. The polypeptide of alternative 33, 34, or 36, wherein the polypeptide shares or comprises at least 90%, 91%, 92%, 93%, 94%, 95%, 96%, 97%, 98%, 99%, or 100% homology or sequence identity to any one or more of SEQ ID NO: 26-27.

38. The polypeptide of alternative 36, wherein the RBD polypeptide is an RBD tandem repeat single chain dimer polypeptide.

39. The polypeptide of alternative 38, wherein the RBD tandem repeat single chain dimer polypeptide comprises a K417N, N439K, E484K, or N501Y mutation with reference to the full S protein (e.g., as set forth in NCBI Accession No. YP_009724390), or any combination thereof.

40. The polypeptide of alternative 38 or 39, wherein the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NOs: 46, or 52-55.

41. The polypeptide of any one of alternatives 38-40, wherein the polypeptide shares or comprises at least 90%, 91%, 92%, 93%, 94%, 95%, 96%, 97%, 98%, 99%, or 100% homology or sequence identity to SEQ ID NO: 41.

42. The polypeptide of alternative 33, 34, or 36, further comprising an N-terminal IgE leader polypeptide sequence.

43. The polypeptide of alternative 42, wherein the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44.

44. The polypeptide of alternative 42 or 43, wherein the RBD polypeptide is an RBD tandem repeat single chain dimer polypeptide.

45. The polypeptide of alternative 44, wherein the RBD tandem repeat single chain dimer polypeptide comprises a K417N, N439K, E484K, or N501Y mutation with reference to the full S protein (e.g., as set forth in NCBI Accession No. YP_009724390), or any combination thereof.

46. The polypeptide of alternative 44 or 45, wherein the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 46, or 52-55.

47. The polypeptide of any one of alternatives 42-46, wherein the polypeptide shares or comprises at least 90%, 91%, 92%, 93%, 94%, 95%, 96%, 97%, 98%, 99%, or 100% homology or sequence identity to any one or more of SEQ ID NO: 42, 64-67, or 69.

48. The polypeptide of alternative 42-43, wherein the RBD polypeptide comprises three tandem copies of RBD.

49. The polypeptide of alternative 48, wherein the three tandem copies of RBD each comprise a K417N, N439K, E484K, or N501Y mutations with reference to the full S protein (e.g., as set forth in NCBI Accession No. YP_009724390), or any combination thereof, or none of these mutations.

50. The polypeptide of alternative 48 or 49, wherein the polypeptide shares or comprises at least 90%, 91%, 92%, 93%, 94%, 95%, 96%, 97%, 98%, 99%, or 100% homology or sequence identity to SEQ ID NO: 68.

51. The polypeptide of alternative 33, wherein the at least one SARS-CoV-2 polypeptide sequence comprises an RBD polypeptide sequence and an M polypeptide sequence.

52. The polypeptide of alternative 33 or 51, wherein the polypeptide shares or comprises at least 90%, 91%, 92%, 93%, 94%, 95%, 96%, 97%, 98%, 99%, or 100% homology or sequence identity to SEQ ID NO: 28.

53. The polypeptide of alternative 33, wherein the at least one SARS-CoV-2 polypeptide sequence comprises a spike (S) polypeptide and an NP polypeptide.

54. The polypeptide of alternative 52, wherein the S polypeptide comprises one or more mutations that improve expression, solubility, and/or immunogenicity.

55. The polypeptide of alternative 53 or 54, wherein the S polypeptide comprises a K968P or V987P mutation with reference to the full S protein (e.g., as set forth in NCBI Accession No. YP_009724390), or both.

56. The polypeptide of any one of alternatives 53-55, wherein the S polypeptide shares or comprises at least 90%, 91%, 92%, 93%, 94%, 95%, 96%, 97%, 98%, 99%, or 100% homology or sequence identity to SEQ ID NO: 56.

57. The polypeptide of any one of alternatives 53-56, further comprising an N-terminal IgE leader polypeptide sequence.

58. The polypeptide of alternative 57, wherein the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44.

59. The polypeptide of any one of alternatives 53-58, wherein the polypeptide shares or comprises at least 90%, 91%, 92%, 93%, 94%, 95%, 96%, 97%, 98%, 99%, or 100% homology or sequence identity to SEQ ID NO: 70.

60. A polypeptide comprising at least one SARS-CoV-2 polypeptide sharing or comprising at least 90%, 91%, 92%, 93%, 94%, 95%, 96%, 97%, 98%, 99%, or 100% homology or sequence identity to any one or more of SEQ ID NO: 29-31, 34-36, 74, or 76.

61. A polypeptide comprising at least one SARS-CoV-2 polypeptide and at least one HDAg polypeptide.

62. The polypeptide of alternative 61, wherein the polypeptide shares or comprises at least 90%, 91%, 92%, 93%, 94%, 95%, 96%, 97%, 98%, 99%, or 100% homology or sequence identity to SEQ ID NO: 32.

63. The polypeptide of alternative 62, further comprising at least one P2A autocatalytic polypeptide cleavage site.

64. The polypeptide of alternative 61 or 63, wherein the polypeptide shares or comprises at least 90%, 91%, 92%, 93%, 94%, 95%, 96%, 97%, 98%, 99%, or 100% homology or sequence identity to SEQ ID NO: 33.

65. The nucleic acid of any one of alternatives 1-32 for use in a medicament, such as for the prevention, treatment or inhibition of SARS-CoV-2 in a subject, preferably a human.

66. The polypeptide of any one of alternatives 33-64 for use in a medicament, such as for the prevention, treatment or inhibition of SARS-CoV-2 in a subject, preferably a human.

67. The polypeptide of any one of alternatives 33-64 or 66, wherein the polypeptide is recombinantly expressed.

68. The polypeptide of alternative 67, wherein the polypeptide is recombinantly expressed in a mammalian, bacterial, yeast, insect, or cell-free system.

69. An immunogenic composition or product combination comprising:
(a) a nucleic acid comprising at least one nucleic acid sequence encoding a SARS-CoV-2 polypeptide; or
(b) a polypeptide comprising at least one SARS-CoV-2 polypeptide, or both.

70. The immunogenic composition or product combination of alternative 69, wherein the at least one nucleic acid sequence encoding a SARS-CoV-2 polypeptide comprises:
i) a nucleic acid sequence encoding an RBD polypeptide;
ii) a nucleic acid sequence encoding an NP polypeptide;
iii) a nucleic acid sequence encoding an M polypeptide;
iv) a nucleic acid sequence encoding an HDAg polypeptide;
v) a nucleic acid sequence encoding a P2A autocatalytic polypeptide cleavage site;
vi) a nucleic acid sequence encoding an IgE leader polypeptide; or
vii) a nucleic acid sequence encoding an S polypeptide;
or any combination thereof.

71. The immunogenic composition or product combination of alternative 69 or 70, wherein the nucleic acid is the nucleic acid of any one of alternatives 1-32.

72. The immunogenic composition or product combination of any one of alternatives 69-71, wherein the nucleic acid shares or comprises at least 90%, 91%, 92%, 93%, 94%, 95%, 96%, 97%, 98%, 99%, or 100% homology or sequence identity to any one or more of SEQ ID NO: 1-12, which is optionally used in a medicament, such as for the prevention, treatment, or inhibition of SARS-CoV-2 in a subject, such as a mammal, preferably a human.

73. The immunogenic composition or product combination of any one of alternatives 69-71, wherein the nucleic acid is codon optimized for expression in a human.

74. The immunogenic composition or product combination of alternative 73, wherein the nucleic acid shares or comprises at least 90%, 91%, 92%, 93%, 94%, 95%, 96%, 97%, 98%, 99%, or 100% homology or sequence identity to any one or more of SEQ ID NO: 13-24, 39-40, 57-63, 71, 73, or 75, which is optionally used in a medicament, such as for the prevention, treatment, or inhibition of SARS-CoV-2 in a subject, such as a mammal, preferably a human.

75. The immunogenic composition or product combination of any one of alternatives 69-74, wherein the at least one SARS-CoV-2 polypeptide comprises:
i) an RBD polypeptide sequence;
ii) an NP polypeptide sequence;
iii) an M polypeptide sequence;
iv) an HDAg polypeptide sequence;
v) a P2A autocatalytic polypeptide cleavage site sequence;
vi) an IgE leader polypeptide sequence; or
vii) an S polypeptide sequence;
or any combination thereof.

76. The immunogenic composition or product combination of any one of alternatives 69-75, wherein the polypeptide is the polypeptide of any one of alternatives 33-64.

77. The immunogenic composition or product combination of any one of alternatives 69-76, wherein the polypeptide shares or comprises at least 90%, 91%, 92%, 93%, 94%, 95%, 96%, 97%, 98%, 99%, or 100% homology or sequence identity to any one or more of SEQ ID NO: 25-36, 4142, 64-70, 72, 74, or 76, which is optionally used in a medicament, such as for the prevention, treatment, or inhibition of SARS-CoV-2 in a subject, such as a mammal, preferably a human.

78. The immunogenic composition or product combination of any one of alternatives 69-77, wherein the polypeptide is recombinantly expressed.

79. The immunogenic composition or product combination of alternative 78, wherein the polypeptide is recombinantly expressed in a mammalian, bacterial, yeast, insect, or cell-free system.

80. The immunogenic composition or product combination of any one of alternatives 69-79, further comprising an adjuvant.

81. The immunogenic composition or product combination of alternative 80, wherein the adjuvant is alum and/or QS21.

82. The immunogenic composition or product combination of any one of alternatives 69-81, wherein the nucleic acid is provided in a recombinant vector.

83. A method of generating an immune response and/or generating neutralizing antibodies in a subject using the immunogenic composition or product combination set forth in any one of alternatives 69-82, comprising:
a) administering to the subject at least one prime dose comprising the nucleic acid; and
b) administering to the subject at least one boost dose comprising the polypeptide.

84. The method of alternative 83, wherein the at least one boost dose further comprises an adjuvant.

85. The method of alternative 84, wherein the adjuvant is alum and/or QS21.

86. The method of any one of alternatives 83-85, wherein the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks.

87. The method of any one of alternatives 83-86, wherein the administration is provided enterally, orally, intranasally, parenterally, subcutaneously, intramuscularly, intradermally, or intravenously or any combination thereof, and optionally with in vivo electroporation.

88. The method of any one of alternatives 83-87, wherein the administration is performed in conjunction with an antiviral therapy.

89. The method of alternative 88, wherein the antiviral therapy comprises administration of dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof.

90. An immunogenic composition or product combination for use in the treatment or inhibition of SARS-CoV-2, comprising:
(a) a nucleic acid comprising at least one nucleic acid sequence encoding a SARS-CoV-2 polypeptide; or
(b) a polypeptide comprising at least one SARS-CoV-2 polypeptide, or both.

91. The immunogenic composition or product combination for use in the treatment or inhibition of SARS-CoV-2 of alternative 90, wherein the at least one nucleic acid sequence encoding a SARS-CoV-2 polypeptide comprises:
i) a nucleic acid sequence encoding an RBD polypeptide;
ii) a nucleic acid sequence encoding an NP polypeptide;
iii) a nucleic acid sequence encoding an M polypeptide;
iv) a nucleic acid sequence encoding an HDAg polypeptide;
v) a nucleic acid sequence encoding a P2A autocatalytic polypeptide cleavage site;
vi) a nucleic acid sequence encoding an IgE leader polypeptide; or
vii) a nucleic acid sequence encoding a S polypeptide;
or any combination thereof.

92. The immunogenic composition or product combination for use in the treatment or inhibition of SARS-CoV-2 of alternative 91, wherein the nucleic acid is the nucleic acid of any one of alternatives 1-32.

93. The immunogenic composition or product combination for use in the treatment or inhibition of SARS-CoV-2 of any one of alternatives 90-92, wherein the nucleic acid shares or comprises at least 90%, 91%, 92%, 93%, 94%, 95%, 96%, 97%, 98%, 99%, or 100% homology or sequence identity to any one or more of SEQ ID NO: 1-12.

94. The immunogenic composition or product combination for use in the treatment or inhibition of SARS-CoV-2 of any one of alternatives 90-92, wherein the nucleic acid is codon optimized for expression in a human.

95. The immunogenic composition or product combination for use in the treatment or inhibition of SARS-CoV-2 of alternative 94, wherein the nucleic acid shares or comprises at least 90%, 91%, 92%, 93%, 94%, 95%, 96%, 97%, 98%, 99%, or 100% homology or sequence identity to any one or more of SEQ ID NO: 13-24, 39-40, 57-63, 71, 73, or 75.

96. The immunogenic composition or product combination for use in the treatment or inhibition of SARS-CoV-2 of any one of alternatives 90-95, wherein the at least one SARS-CoV-2 polypeptide comprises;
i) an RBD polypeptide sequence;
ii) an NP polypeptide sequence;
iii) an M polypeptide sequence.
iv) an HDAg polypeptide sequence;
v) a P2A autocatalytic polypeptide cleavage site sequence;
vi) an IgE leader polypeptide sequence; or
vii) an S polypeptide sequence;
or any combination thereof.

97. The immunogenic composition or product combination for use in the treatment or inhibition of SARS-CoV-2 of any one of alternatives 90-96, wherein the polypeptide is the polypeptide of any one of alternatives 33-64.

98. The immunogenic composition or product combination for use in the treatment or inhibition of SARS-CoV-2 of any one of alternatives 90-97, wherein the polypeptide shares or comprises at least 90%, 91%, 92%, 93%, 94%, 95%, 96%, 97%, 98%, 99%, or 100% homology or sequence identity to any one or more of SEQ ID NO: 25-36, 41-42, 64-70, 72, 74, or 76.

99. The immunogenic composition or product combination for use in the treatment or inhibition of SARS-CoV-2 of any one of alternatives 90-98, wherein the polypeptide is recombinantly expressed.

100. The immunogenic composition or product combination for use in the treatment or inhibition of SARS-CoV-2 of alternative 99, wherein the polypeptide is recombinantly expressed in a mammalian, bacterial, yeast, insect, or cell-free system.

101. The immunogenic composition or product combination for use in the treatment or inhibition of SARS-CoV-2 of any one of alternatives 90-100, further comprising an adjuvant.

102. The immunogenic composition or product combination for use in the treatment or inhibition of SARS-CoV-2 of alternative 101, wherein the adjuvant is alum and/or QS21.

103. The immunogenic composition or product combination for use in the treatment or inhibition of SARS-CoV-2 of any one of alternatives 90-102, wherein the nucleic acid is provided in a recombinant vector.

104. A nucleic acid comprising, consisting essentially of, or consisting of at least one SARS-CoV-2 nucleic acid component joined to a nucleic acid encoding an IgE leader sequence, preferably a nucleic acid encoding the amino acid sequence MDWTWILFLVAAATRVHS (SEQ ID NO: 44), or an IgE leader nucleic acid sequence sharing or comprising at least 90%, 91%, 92%, 93%, 94%, 95%, 96%, 97%, 98%, 99%, or 100% homology or sequence identity to SEQ ID NO: 43.

105. Use of the nucleic acid of alternative 104 or a protein encoded thereby as a medicament, including a medicament that treats or inhibits a SARS-CoV-2 infection.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts exemplary recombinant immunogenic compositions that can be used as medicaments such as for the prevention, treatment, or inhibition of SARS-CoV-2 in a subject, for example utilizing a heterologous prime-boost approach. Any of the exemplary compositions shown herein may be used for any of the methods or uses disclosed herein.

FIG. 2 depicts additional exemplary recombinant immunogenic compositions that can be used as medicaments such as for the prevention, treatment, or inhibition of SARS-CoV-2, including different variants, in a subject, for example utilizing a heterologous prime-boost approach. Any of the exemplary compositions shown herein may be used for any of the methods or uses disclosed herein.

FIGS. 3A-B depict immunization of BALB/c and C57BL/6 mice using exemplary SARS-CoV-2 constructs disclosed herein. FIG. 3A shows end point ELISA of mice serum against RBD and S protein. FIG. 3B shows in vitro SARS-CoV-2 viral neutralization using serum from immunized mice.

FIG. 4 depicts T cell response of immunized mice against peptide pools covering the SARS-CoV-2 RBD, M, and NP proteins as detected by ELISpot.

FIG. 5A depicts anti-S protein antibody titers in mice immunized with a prime/boost approach using OC-2.3 DNA and recombinant S protein with QS21 adjuvant (rS/QS21). The combinations tested were: 1) OC-2.3 DNA prime and rS/QS21 protein boost; 2) OC-2.3 DNA prime and OC-2.3 DNA boost, 3) rS/QS21 protein prime and rS/QS21 protein boost; and 4) rS/QS21 protein prime and OC-2.3 DNA boost.

FIG. 5B depicts T cell response from mice immunized with the prime/boost approach of FIG. 5A against peptide pools covering the SARS-CoV-2 RBD, M, or NP proteins, or the full length RBD, M, or NP proteins.

FIG. 6A depicts anti-S protein antibody titers in rabbits immunized with OC-2.3 DNA tested two weeks after either the first dose (at week 2) or the second dose (at week 5), and administered either 500, 1000, or 1500 μg of the DNA.

FIG. 6B depicts anti-S or anti-NP (N) protein antibody titers in cynomolgus macaques immunized with OC-2.3 DNA tested at either week 0 or week 5 after two 1000 μg doses.

FIG. 6C depicts quantification of SARS-CoV-2 RNA in cynomolgus macaques immunized with either OC-2.3 DNA or control DNA at days 4 or 20 following a SARS-CoV-2 challenge.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Definitions

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless defined otherwise, all technical and scientific terms used herein have the same meaning as is commonly understood by one of ordinary skill in the art to which this disclosure belongs. If there is a plurality of definitions for a term herein, those in this section prevail unless stated otherwise. The practice of the present disclosure will employ, unless indicated specifically to the contrary, conventional methods of molecular biology and recombinant DNA techniques within the skill of the art, many of which are described below for the purpose of illustration.

The terms “individual”, “subject”, or “patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

Some embodiments described herein relate to pharmaceutical compositions that comprise, consist essentially of, or consist of an effective amount of an oligonucleotide, a protein, or both, described herein and a pharmaceutically acceptable carrier, excipient, or combination thereof. A pharmaceutical composition described herein is suitable for human and/or veterinary applications.

The terms “function” and “functional” as used herein refer to a biological, enzymatic, or therapeutic function.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The terms “effective amount” or “effective dose” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “pharmaceutically acceptable salts” includes relatively non-toxic, inorganic and organic acid, or base addition salts of compositions, including without limitation, analgesic agents, therapeutic agents, other materials, and the like. Examples of pharmaceutically acceptable salts include those derived from mineral acids, such as hydrochloric acid, sulfuric acid, and those derived from organic acids, such as ethanesulfonic acid, benzenesulfonic acid, p-toluenesulfonic acid, and the like. Examples of suitable inorganic bases for the formation of salts include phosphates, hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; or trihydroxymethyl aminoethane.

“Formulation”, “pharmaceutical composition”, and “composition” as used interchangeably herein are equivalent terms referring to a composition of matter for administration to a subject.

The term “pharmaceutically acceptable” means compatible with therapy for a subject, and in particular, a human.

The terms “agent” refers to an active agent that has biological activity and may be used in a therapy. Also, an “agent” can be synonymous with “at least one agent,” “compound,” or “at least one compound,” and can refer to any form of the agent, such as a derivative, analog, salt or a prodrug thereof. The agent can be present in various forms, components of molecular complexes, and pharmaceutically acceptable salts (e.g., hydrochlorides, hydrobromides, sulfates, phosphates, nitrates, borates, acetates, maleates, tartrates, and salicylates). The term “agent” can also refer to any pharmaceutical molecules or compounds, therapeutic molecules or compounds, matrix forming molecules or compounds, polymers, synthetic molecules and compounds, natural molecules and compounds, and any combination thereof.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral, intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration. The pharmaceutical compositions described herein can also be administered to subjects along with other therapies, such as T cells, Natural Killer cells, B cells, macrophages, lymphocytes, stem cells, bone marrow cells, or hematopoietic stem cells.

The pharmaceutical compound can also be administered in a local rather than systemic manner, for example, via injection of the compound directly into an organ, tissue, or infected area, often in a depot or sustained release formulation. Furthermore, one may administer the compound in a targeted drug delivery system, for example, in a liposome coated with a tissue specific antibody. The liposomes may be targeted to and taken up selectively by the organ, tissue, cancer, tumor, or infected area.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the liver.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the osmolarity and/or composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties, which may be incorporated into any one or more of the formulations set forth herein, include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with an immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants, which can be included in any one or more of the formulations set forth herein include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

Some embodiments disclosed herein related to selecting a subject or patient in need. In some embodiments, a patient is selected who is in need of immunogenicity against a viral infection such as SARS-CoV-2. In some embodiments, a patient is selected as one identified as having a SARS-CoV-2 infection or as one in need of treatment of a viral infection such as SARS-CoV-2. In some embodiments, a patient is selected who has previously been treated for a viral infection, such as SARS-CoV-2. In some embodiments, a patient is selected who has previously been treated for being at risk of a viral infection, such as SARS-CoV-2. In some embodiments, a patient is selected who has developed a recurrence of a viral infection, such as SARS-CoV-2. In some embodiments, a patient is selected who has developed resistance to therapies for a viral infection, such as SARS-CoV-2. In some embodiments, a patient is selected who may have any combination of the aforementioned selection criteria. Such selections can be made by clinical and diagnostic evaluation of the subject or a combination of both.

The terms “treat”, “treating”, “treatment”, “therapeutic”, or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein can in some but not all contexts include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from or developing a disease or condition.

The term “inhibit” as used herein has its ordinary meaning as understood in light of the specification, and may refer to the reduction of a viral infection, such as SARS-CoV-2. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

The term “immunogenic composition” as used herein refers to a substance or mixture of substances, including but not limited to antigens, epitopes, nucleic acids, peptides, polypeptides, proteins, polysaccharides, lipids, haptens, toxoids, inactivated organisms, or attenuated organisms, or any combination thereof, intended to elicit an immune response when administered to a host. The immune response includes both an innate and adaptive immune response, the latter of which establishes a lasting immunological memory through cells such as memory T cells and memory B cells. The antibodies created during the initial immune response to the immunogenic composition can be produced in subsequent challenges of the same antigens, epitopes, nucleic acids, peptides, polypeptides, proteins, polysaccharides, lipids, haptens, toxoids, inactivated organisms, or attenuated organisms, or a live organism or pathogen that exhibits the antigens, epitopes, nucleic acids, peptides, polypeptides, proteins, polysaccharides, lipids, haptens, or toxoids or any combination thereof. In this manner, the immunogenic composition may serve as a vaccine against a specific pathogen. Immunogenic compositions may also include one or more adjuvants to stimulate the immune response and increase the efficacy of protective immunity.

The term “product combination” as used herein refers to set of two or more individual compounds, substances, materials, or compositions that can be used together for a unified function. In some embodiments, a product combination comprises at least one nucleic acid composition and at least one polypeptide composition that are used together to elicit an immune response when administered to a host, optionally to a greater degree than would be elicited if only one composition type were to be administered.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Programs containing algorithms for codon optimization are known to those skilled in the art. Programs can include, for example, OptimumGene, GeneGPS® algorithms, etc. Additionally, synthetic codon optimized sequences can be obtained commercially for example from Integrated DNA Technologies and other commercially available DNA sequencing services. Those skilled in the art will appreciate that gene expression levels are dependent on many factors, such as promoter sequences and regulatory elements. As noted for most bacteria, small subsets of codons are recognized by tRNA species leading to translational selection, which can be an important limit on protein expression. In this aspect, many synthetic genes can be designed to increase their protein expression level.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

In some embodiments, the nucleic acid or peptide sequences presented herein and used in the examples are functional in various biological systems including but not limited to humans, mice, rabbits, E. coli, yeast, and mammalian cells. In other embodiments, nucleic acid or peptide sequences sharing at least or lower than 0%, 10%, 20%, 30%, 40%, 50%, 60%, 70%, 80%, 90%, 91%, 92%, 93%, 940%, 95%, 96%, 97%, 98%, 99%, or 100% similarity, or any percentage within a range defined by any two of the aforementioned percentages similarity to the nucleic acid or peptide sequences presented herein and used in the examples can also be used with no effect on the function of the sequences in biological systems. As used herein, the term “similarity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% similarity can encode for the same polypeptide by comprising different codons that encode for the same amino acid during translation.

The term “recombinantly expressed” as used herein refers to the production of proteins in optimized or adapted biological systems. These systems provide advantages over protein expression in a natural host, including but not limited to high expression (overexpression), ease of purification, ease of transformation, inducibility, low cost, or stability of the protein. In some embodiments, proteins are expressed in mammalian, bacteria, yeast, insect, or cell-free recombinant expression systems. Each system has its own advantages or disadvantages. For example, bacterial expression systems are highly optimized for overexpression, but may cause misfolding or aggregation of the produced protein, yeast systems are useful when post-translational modifications are necessary, and insect and mammalian systems are useful for proper RNA splicing that occurs in higher-order organisms. In some embodiments, recombinant polypeptides are produced and purified from mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sf9, or S. frugiperda Sf21 cells, or in a cell-free system. In some embodiments, expression genes, vectors, or constructs are delivered to the recombinant expression systems in the form of plasmids, bacteriophages, viruses, adeno-associated viruses (AAVs), baculovirus, cosmids, fosmids, phagemids, BACs, YACs, or HACs. For more discussion on recombinant expression systems, see Gomes et al. “An Overview of Heterologous Expression Host Systems for the Production of Recombinant Proteins” ((2016) Adv. Anim. Vet. Sci. 4(7):346-356), hereby expressly incorporated by reference in its entirety.

The term “coronavirus” as used herein refers to the family of enveloped, positive-sense, single stranded RNA viruses that infect mammals and birds. In humans, coronavirus infections can cause mild symptoms as a common cold, or more severe respiratory conditions such as severe acute respiratory syndrome (SARS), acute respiratory distress syndrome (ARDS), coughing, congestion, sore throat, shortness of breath, pneumonia, bronchitis, and hypoxia. Other symptoms include but are not limited to fever, fatigue, myalgia, and gastrointestinal symptoms such as vomiting, diarrhea, and abdominal pain. The viral envelope comprises spike (“S”), envelope (“E”), membrane (“M”), and hemagglutinin esterase (“HE”) transmembrane structural proteins. The S protein comprises a receptor binding domain (“RBD”), a highly immunogenic region that determines the host receptor specificity of the virus strain. The viral nucleocapsid comprises multiple nucleocapsid (“N” or “NP”) proteins coating the RNA genome. During infection, the S protein attaches to a host cell receptor and initiate entry into the host cell through endocytosis or fusion of the envelope membrane. The RNA genome is translated by the host ribosome to produce new structural proteins and RNA-dependent RNA polymerases, which replicate the viral genome. Viral particles are assembled in the host endoplasmic reticulum and are shed by Golgi-mediated exocytosis. More information about the structure and infection cycle of coronaviruses can be found in Fehr A R & Perlman S. “Coronaviruses: An Overview of Their Replication and Pathogenesis” Methods Mol. Biol. (2015); 1282:1-23, hereby expressly incorporated by reference in its entirety.

The terms “SARS-CoV-2” and “2019-nCoV” as used herein refers to the coronavirus strain or strains responsible for the human coronavirus disease 2019 (COVID-19) pandemic. The contagiousness, long incubation period, and modern globalization has led to worldwide spread of the virus. Development of SARS and other respiratory issues in infected individuals has resulted in immense stress on medical infrastructure. Treatments and vaccines for SARS-CoV-2 and other coronaviruses in humans are starting to be approved, but additional testing is necessary. Reference sequences are available by NCBI GenBank accession number: MN908947.3 (e.g. complete genome), YP_009724390 (e.g. surface glycoprotein), YP_009724393.1 (e.g. membrane glycoprotein), and YP_009724397.2 (e.g. nucleocapsid phosphoprotein). Like the original SARS virus (SARS-CoV-1), SARS-CoV-2 infects human cells by binding to angiotensin-converting enzyme 2 (ACE2) through the RBD of the S protein. The RBD, M protein, and NP protein are good candidates for the development of treatments, prophylaxes, interventions, vaccines, or immunogenic compositions against SARS-CoV-2 and other coronaviruses. The embodiments disclosed herein can be applied to other coronaviruses, including but not limited to HCoV-229E, HCoV-OC43, SARS-CoV-1, HCoV NL63, HCoV-HKU1, and MERS-CoV.

During the COVID-19 pandemic, emergent genetic variants were discovered. These variants may exhibit different host specificity or increased transmissibility, infectivity, and/or virulence. Furthermore, there are concerns that these variants or new variants may reduce the efficacy of currently approved vaccines. The primary genetic mutations of concern involve the S protein (and corresponding RBD), which the virus uses for host receptor binding; as the current vaccines are directed to immunogenicity against these S proteins, they may result in reduced efficacy against these mutant strains. Three prominent variants are the strain first identified in the United Kingdom (20B/501Y.V1, VOC 20212/01, B.1.1.7), the strain first identified in South Africa (20C/501Y.V2, B.1.351), and the Brazilian variant first identified in Japan (20J/501Y.V3, P.1). These variants have been found to exhibit rapid and wide-spread transmission throughout the world. A common mutation among these three strains is N501Y, which is at one of six contact residues of the RBD that interfaces with human ACE2 and has been shown to increase affinity towards ACE2 (Starr et al. “Deep Mutational Scanning of SARS-CoV-2 Receptor Binding Domain Reveals Constraints on Folding and ACE2 Binding” Cell; (2020) 182(5); 1295-1310, hereby expressly incorporated by reference in its entirety). The South African variant also comprises the mutations K417N and E484K. The Brazilian variant has 17 unique amino acid changes and three deletions, including K417T, E484K, and N501Y mutations in the spike protein receptor binding domain. Other variants comprise the N439K mutation. These mutations have been suspected to interfere with antibody recognition. As disclosed herein, in some embodiments, the nucleic acids and polypeptides for use as immunogenic compositions may encode or comprise these mutations, or other mutations within the S protein or corresponding RBD. The incorporation of these immunogens into the formulations and methods described herein will produce an increased diversity of antibody and T cell response in the inoculated patient, which will provide for a robust protection against SARS-CoV-2 and SARS-CoV-2 variants.

In some embodiments, the RBD sequences used herein are tandem repeat single chain dimer variants. RBD dimers have been shown to improve immunogenicity and increase neutralizing antibody titers. Both disulfide-linked dimers and single chain (covalently linked) dimers are effective in this aspect. In some embodiments, the RBD tandem repeat single chain dimer is constructed by fusing two coronavirus RBD sequences with or without additional linkers or other amino acids. An example of an RBD tandem repeat single chain dimer polypeptide is embodied in SEQ ID NO: 46. An example of a nucleic acid sequence encoding an RBD tandem repeat single chain dimer polypeptide is embodied in SEQ ID NO: 45. In some embodiments, the RBD tandem repeat single chain dimers may comprise any one or more of the mutations disclosed herein and/or additional mutations associated with one or more SARS-CoV-2 variants. For example, the RBD tandem repeat single chain dimer may comprise a K417N, N439K, E484K, or N501Y mutation, or any combination thereof, or none of these mutations, associated with a SARS-CoV-2 variant (where it is understood that these mutations are set forth with reference to the full S protein (e.g., as set forth in NCBI Accession No. YP_009724390)). Throughout this disclosure, RBD tandem repeat single chain dimers may also be referred as RBD version 2 (RBDv2). Additional insight into RBD tandem repeat single chain dimers may be found in Dai et al. “A Universal Design of Betacoronavirus Vaccines against COVID-19, MERS, and SARS” Cell. (2020); 182(3): 722-733, which is hereby expressly incorporated by reference in its entirety.

In some embodiments, the RBD sequences are assembled in multimeric variants, such as variants with 3, 4, 5, 6, 7, 8, 9, or 10 copies of one or more RBD sequences. In some embodiments, the RBD sequences are assembled into trimeric variants. An example of a construct with a trimeric RBD variants is OC-2.4. In some embodiments, each of the RBD sequences in the multimeric variants may comprise any one or more of the mutations disclosed herein and/or additional mutations associated with one or more SARS-CoV-2 variants. For example, one or more RBD sequences in the multimeric variants may comprise a K417N, N439K, E484K, or N501Y mutation, or any combination thereof, or none of these mutations, associated with a SARS-CoV-2 variant (where it is understood that these mutations are set forth with reference to the full S protein (e.g., as set forth in NCBI Accession No. YP_009724390)).

The terms “autocatalytic peptide cleavage site” or “2A peptide” as used herein refer to a peptide sequence that undergo cleavage of a peptide bond between two constituent amino acids, resulting in separation of the two proteins that flank the sequence. The cleavage is believed to be a result of a ribosomal “skipping” of the peptide bond formation between the C-terminal proline and glycine in the 2A peptide sequence. Four autocatalytic peptide cleavage site sequences identified to date have seen substantial use in biomedical research: foot-and-mouth disease virus 2A (F2A); equine rhinitis A virus (ERAV) 2A (E2A); porcine teschovirus-1 2A (P2A), and Thosea asigna virus 2A (T2A). In some embodiments, the P2A autocatalytic peptide cleavage site nucleic acid (SEQ ID NO: 37) and polypeptide (SEQ ID NO: 38) sequences are used. In some embodiments, the P2A nucleic acid or polypeptide used can be substituted with an F2A, E2A, or T2A nucleic acid or polypeptide.

In some embodiments, the nucleic acids or peptides used herein comprise sequences representing hepatitis D antigen (HDAg) variants. Hepatitis D is a virusoid that relies on hepatitis B coinfection or superinfection to replicate. The circular single-stranded RNA of hepatitis D is amplified using host RNA polymerases, but also contains a single hepatitis D antigen (HDAg) gene. During hepatitis B and D coinfection or superinfection, intact hepatitis D viruses are packaged with an envelope containing hepatitis B surface antigens surrounding the RNA genome that is coated with HDAg protein. Incorporation of the hepatitis B surface antigens is essential for hepatitis D infectivity, as hepatitis D does not encode its own receptor binding proteins. Coinfection or superinfection with hepatitis D causes more severe complications, with increased risk of liver failure, cirrhosis, and cancer. A small (24 kDa) and large (27 kDa, 213 amino acids excluding the start methionine) isoform exist for HDAg and are translated from the same open reading frame on the HDV genome. Deamination of the adenosine in a UAG stop codon at codon 196 of the coding sequence allows for translation to continue and produce the large isoform. Unless expressly stated otherwise, the embodiments described herein comprise the large isoform of HDAg. In some embodiments, the HDAg sequences comprise at least one of four different HDAg strain sequences: “HDAg genotype 1A”, “HDAg genotype 1B”, “HDAg genotype 2A”, or “HDAg genotype 2B”. Additional information about HDAg sequences and uses thereof can be found in PCT Publication WO 2017/132332, hereby expressly incorporated by reference in its entirety.

The term “IgE leader sequence” as used herein refers to the amino acid sequence MDWTWILFLVAAATRVHS (SEQ ID NO: 44), which can be appended to the N-terminus of a protein to both enhance translation and increase immunogenicity. Translation is particularly upregulated when the IgE leader sequence is used in combination with a functional Kozak sequence. An exemplary embodiment of a nucleic acid sequence that encodes for the amino acid IgE leader sequence is represented as SEQ ID NO: 43. However, it would be clearly apparent to one skilled in the art to develop alternative nucleic acid sequence that would result in the same amino acid sequence when translated. Additional insight into the use of an IgE leader sequence may be found in Vijayachari et al. “Immunogenicity of a novel enhanced consensus DNA vaccine encoding the leptospiral protein LipL45” Hum. Vaccin. Immunother. (2015):11(8):1945-53, which is hereby expressly incorporated by reference in its entirety.

The terms “in vivo electroporation”, “electroporation”, and “EP” as used herein refers to the delivery of genes, nucleic acids, DNA, RNA, proteins, or vectors into cells of living tissues or organisms using electrical currents using techniques known in the art. Electroporation can be used as an alternative to other methods of gene transfer such as viruses (transduction), lipofection, gene gun (biolistics), microinjection, vesicle fusion, or chemical transformation. Electroporation limits the risk of immunogenicity and detrimental integration or mutagenesis of the cell genome. DNA vectors such as plasmids are able to access the cell nucleus, enabling transcription and translation of constituent genes. In some embodiments, the genes, nucleic acids, DNA, RNA, proteins, or vectors are added to the target tissue or organism by subcutaneous, intramuscular, or intradermal injection. An electroporator then delivers short electrical pulses via electrodes placed within or proximal to the injected sample. As used herein, the term “im/EP” refers to in vivo electroporation of a sample delivered intramuscularly (“im”).

The terms “KI8-hACE2” or “B6.Cg-Tg(KI8-ACE2)2Prlmn/J” as used herein refers to a transgenic mouse model expressing human ACE2, the receptor that coronaviruses such as SARS-CoV-1 and SARS-CoV-2 used to infect human cells. Expression of human ACE2 is driven by the human cytokeratin 18 promoter. These mice can be used as experimental models for SARS-CoV-2 viral infections. Other similar mouse models can be used as alternatives.

Where a range of values is provided, it is understood that the upper and lower limit, and each intervening value between the upper and lower limit of the range is encompassed within the embodiments.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

### Exemplary Immunogenic Composition Embodiments

Disclosed herein are nucleic acids that can be used as immunogenic compositions or part of immunogenic product combinations, for example, to generate an immune response against SARS-CoV-2 or other coronavirus, and/or generate neutralizing antibodies against SARS-CoV-2 or other coronavirus in a subject.

In some embodiments, the nucleic acid comprises at least one nucleic acid sequence encoding a SARS-CoV-2 polypeptide and at least one nucleic acid sequence encoding a P2A autocatalytic polypeptide cleavage site. In some embodiments, the at least one nucleic acid sequence encoding a SARS-CoV-2 polypeptide comprises a nucleic acid sequence encoding a receptor binding domain (RBD) polypeptide and a nucleic acid encoding a nucleoprotein (NP) polypeptide. In some embodiments, the nucleic acid shares or comprises at least 90%, 91%, 92%, 93%, 94%, 95%, 96%, 97%, 98%, 99%, or 100% homology or sequence identity to SEQ ID NO: 1 or 13. In some embodiments, the at least one nucleic acid sequence encoding a SARS-CoV-2 polypeptide comprises a nucleic acid sequence encoding an RBD polypeptide, a nucleic acid sequence encoding an M polypeptide, and a nucleic acid sequence encoding an NP polypeptide. In some embodiments, the nucleic acid shares or comprises at least 90%, 91%, 92%, 93%, 94%, 95%, 96%, 97%, 98%, 99%, or 100% homology or sequence identity to any one of SEQ ID NO: 2-3, or 14-15. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation, or any combination thereof, or none of these mutations with reference to the full S protein (e.g., as set forth in NCBI Accession No. YP_009724390)). In some embodiments, the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to SEQ ID NO: 39. In some embodiments, the RBD polypeptide comprises three tandem copies of RBD (or RBDv2). In some embodiments, the three tandem copies of RBD each comprise a K417N, N439K, E484K, or N501Y mutation with reference to the full S protein (e.g., as set forth in NCBI Accession No. YP_009724390), or any combination thereof, or none of these mutations.

As applied to any of the nucleic acids disclosed herein, in some embodiments, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation with reference to the full S protein (e.g., as set forth in NCBI Accession No. YP_009724390)), or any combination thereof, or none of these mutations. In some embodiments, the nucleic acid sequence encoding the RBD tandem repeat single chain dimer polypeptide shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 45, or 47-50. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 40, 57-60, or 62. In some embodiments, the RBD polypeptide comprise three tandem copies of RBD (or RBDv2). In some embodiments, the three tandem copies of RBD each comprise a K417N, N439K, E484K, or N501Y mutation with reference to the full S protein (e.g., as set forth in NCBI Accession No. YP_009724390), or any combination thereof, or none of these mutations. In some embodiments, the nucleic acid shares or comprises at least 90%, 91%, 92%, 93%, 94%, 95%, 96%, 97%, 98%, 99%, or 100% homology or sequence identity to SEQ ID NO: 61.

In some embodiments, the at least one nucleic acid sequence encoding a SARS-CoV-2 polypeptide comprises a nucleic acid sequence encoding an RBD polypeptide and a nucleic acid sequence encoding an M polypeptide. In some embodiments, the nucleic acid shares or comprises at least 90%, 91%, 92%, 93%, 94%, 95%, 96%, 97%, 98%, 99%, or 100% homology or sequence identity to SEQ ID NO: 4 or 16.

In some embodiments, the at least one nucleic acid sequence encoding the SARS-CoV-2 polypeptide comprises a nucleic acid sequence encoding for a spike (S) polypeptide. In some embodiments, the at least one nucleic acid sequence encoding the SARS-CoV-2 polypeptide comprises a nucleic acid sequence encoding for a membrane (M) polypeptide. In some embodiments, the at least one nucleic acid sequence encoding the SARS-CoV-2 polypeptide further comprises a nucleic acid sequence encoding for a nucleoprotein (NP) polypeptide. In some embodiments, the at least one nucleic acid sequence encoding the SARS-CoV-2 polypeptide comprises a nucleic acid sequence encoding for a S polypeptide, a nucleic acid sequence encoding for a M polypeptide, or a nucleic acid sequence encoding for a NP polypeptide, or any combination thereof. In some embodiments, the S polypeptide comprises mutations to facilitate improved expression, solubility, and/or immunogenicity. In some embodiments, the S polypeptide comprises a K968P or V987P mutation with reference to the full S protein (e.g., as set forth in NCBI Accession No. YP_009724390), or both. In some embodiments, the nucleic acid sequence encoding the S polypeptide shares or comprises at least 90%, 91%, 92%, 93%, 94%, 95%, 96%, 97%, 98%, 99%, or 100% homology or sequence identity to SEQ ID NO: 51. In some embodiments, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43. In some embodiments, the nucleic acid shares or comprises at least 90%, 91%, 92%, 93%, 94%, 95%, 96%, 97%, 98%, 99%, or 100% homology or sequence identity to SEQ ID NO: 63.

In some embodiments, the nucleic acid comprises at least one nucleic acid sequence encoding a SARS-CoV-2 polypeptide. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 5-7, 17-19, 22-24, 73, or 75.

In some embodiments, the nucleic acid comprises at least one nucleic acid sequence encoding a SARS-CoV-2 polypeptide and at least one nucleic acid sequence encoding a hepatitis D antigen (HDAg). In some embodiments, the nucleic acid shares or comprises at least 90%, 91%, 92%, 93%, 94%, 95%, 96%, 97%, 98%, 99%, or 100% homology or sequence identity to SEQ ID NO: 8 or 20. In some embodiments, the nucleic acid further comprises at least one nucleic acid sequence encoding a P2A autocatalytic polypeptide cleavage site. In some embodiments, the nucleic acid shares or comprises at least 90°/c, 91%, 92%, 93%, 94%, 95%, 96%, 97%, 98%, 99%, or 100% homology or sequence identity to SEQ ID NO: 9 or 21.

In some embodiments of any one of the nucleic acids disclosed herein, the nucleic acid further comprises a 5′ IgE leader nucleic acid sequence. In some embodiments, the 5′ IgE leader nucleic acid sequence shares or comprises at least 90%, 91%, 92%, 93%, 94%, 95%, 96%, 97%, 98%, 99%, or 100% homology or sequence identity to SEQ ID NO: 43.

In any of the nucleic acids disclosed herein, the nucleic acid may encode for any one or more of the SARS-CoV-2 polypeptides disclosed herein or otherwise conventionally known in the art. In some embodiments, the one or more SARS-CoV-2 polypeptides comprise an RBD polypeptide. In some embodiments, the RBD polypeptide is from the SARS-CoV-2 virus or a variant thereof. In some embodiments, the RBD polypeptide comprises a K417N, N439K, E484K, or N501Y mutation with reference to the full S protein (e.g., as set forth in NCBI Accession No. YP_009724390), or any combination thereof, or none of these mutations. In some embodiments, a nucleic acid encoding for the RBD polypeptide is represented by SEQ ID NO: 10 or 22. In some embodiments, the RBD polypeptide is represented by SEQ ID NO: 34. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation with reference to the full S protein (e.g., as set forth in NCBI Accession No. YP_009724390), or any combination thereof, or none of these mutations. In some embodiments, a nucleic acid encoding for the RBD polypeptide is represented by any one of SEQ ID NOs: 45, or 47-50. In some embodiments, the RBD polypeptide is represented by any one of SEQ ID NOs: 46, or 52-55. In some embodiments, a nucleic acid encoding for an M polypeptide is represented by SEQ ID NOs: 11 or 23. In some embodiments, the M polypeptide is represented by SEQ ID NO: 35. In some embodiments, a nucleic acid encoding for an NP polypeptide is represented by SEQ ID NOs: 12 or 24. In some embodiments, the NP polypeptide is represented by SEQ ID NO: 36.

Any one of the nucleic acids disclosed herein may be used in a medicament or for the manufacture of a medicament. In some embodiments, the medicament is used for the prevention, treatment, or inhibition of SARS-CoV-2 or other coronavirus in a subject. In some embodiments, the subject is a human.

Also disclosed herein are polypeptides that can be used as immunogenic compositions or part of immunogenic product combinations, for example, to generate an immune response against SARS-CoV-2 or other coronavirus, and/or generate neutralizing antibodies against SARS-CoV-2 or other coronavirus in a subject.

In some embodiments, the polypeptide comprises at least one SARS-CoV-2 polypeptide sequence and at least one P2A autocatalytic polypeptide cleavage site. In some embodiments, the at least one SARS-CoV-2 polypeptide sequence comprises an RBD polypeptide sequence and an NP polypeptide sequence. In some embodiments, the polypeptide shares or comprises at least 90%, 91%, 92%, 93%, 94%, 95%, 96%, 97%, 98%, 99%, or 100% homology or sequence identity to SEQ ID NO: 25. In some embodiments, the at least one SARS-CoV-2 polypeptide sequence comprises an RBD polypeptide sequence, an M polypeptide sequence, and an NP polypeptide sequence. In some embodiments, the polypeptide shares or comprises at least 90%, 91%, 92%, 93%, 94%, 95%, 96%, 97%, 98%, 99%, or 100% homology or sequence identity to SEQ ID NO: 26 or 27. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation with reference to the full S protein (e.g., as set forth in NCBI Accession No. YP_009724390), or any combination thereof, or none of these mutations. In some embodiments, the RBD tandem repeat single chain dimer polypeptide shares or comprises at least 90%, 91%, 92%, 93%, 94%, 95%, 96%, 97%, 98%, 99%, or 100% homology or sequence identity to any one of SEQ ID NOs: 46, or 52-55. In some embodiments, the polypeptide shares or comprises at least 90%, 91%, 92%, 93%, 94%, 95%, 96%, 97%, 98%, 99%, or 100% homology or sequence identity to SEQ ID NO: 41. In some embodiments, the RBD polypeptide comprises three tandem copies of RBD (or RBDv2). In some embodiments, the three tandem copies of RBD each comprise a K417N, N439K, E484K, or N501Y mutation with reference to the full S protein (e.g., as set forth in NCBI Accession No. YP_009724390), or any combination thereof, or none of these mutations.

As applied to any of the polypeptides disclosed herein, in some embodiments, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation with reference to the full S protein (e.g., as set forth in NCBI Accession No. YP_009724390), or any combination thereof, or none of these mutations. In some embodiments, the RBD tandem repeat single chain dimer polypeptide shares or comprises at least 90%, 91%, 92%, 93%, 94%, 95%, 96%, 97%, 98%, 99%, or 100% homology or sequence identity to any one of SEQ ID NO: 46, or 52-55. In some embodiments, the polypeptide shares or comprises at least 90%, 91%, 92%, 93%, 94%, 95%, 96%, 97%, 98%, 99%, or 100% homology or sequence identity to any one of SEQ ID NO: 42, 64-67, or 69. In some embodiments, the RBD polypeptide comprises three tandem copies of RBD (or RBDv2). In some embodiments, the three tandem copies of RBD each comprises a K417N, N439K, E484K, or N501Y mutation with reference to the full S protein (e.g., as set forth in NCBI Accession No. YP_009724390), or any combination thereof, or none of these mutations. In some embodiments, the polypeptide shares or comprises at least 90%, 91%, 92%, 93%, 94%, 95%, 96%, 97%, 98%, 99%, or 100% homology or sequence identity to SEQ ID NO: 68.

In some embodiments, the at least one SARS-CoV-2 polypeptide sequence comprises an RBD polypeptide sequence and an M polypeptide sequence. In some embodiments, the polypeptide shares or comprises at least 90%, 91%, 92%, 93%, 94%, 95%, %%, 97%, 98%, 99%, or 100% homology or sequence identity to SEQ ID NO: 28.

In some embodiments, the at least one SARS-CoV-2 polypeptide comprises a spike (S) polypeptide. In some embodiments, the at least one SARS-CoV-2 polypeptide further comprises an NP polypeptide. In some embodiments, the S polypeptide comprises mutations to facilitate improved expression, solubility, and/or immunogenicity. In some embodiments, the S polypeptide comprises a K968P or V987P mutation with reference to the full S protein (e.g., as set forth in NCBI Accession No. YP_009724390), or both. In some embodiments, the S polypeptide shares or comprises at least 90%, 91%, 92%, 93%, 94%, 95%, 96%, 97%, 98%, 99%, or 100% homology or sequence identity to SEQ ID NO: 56. In some embodiments, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the polypeptide shares or comprises at least 90%, 91%, 92%, 93%, 94%, 95%, 96%, 97%, 98%, 99%, or 100% homology or sequence identity to SEQ ID NO: 70.

In some embodiments, the polypeptide comprises at least one SARS-CoV-2 polypeptide sharing or comprising at least 90%, 91%, 92%, 93%, 94%, 95%, 96%, 97%, 98%, 99%, or 100% homology or sequence identity to any one or more of SEQ ID NO: 29-31, 34-36, 74, or 76.

In some embodiments, the polypeptide comprises at least one SARS-CoV-2 polypeptide and at least one HDAg polypeptide. In some embodiments, the polypeptide shares or comprises at least 90%, 91%, 92%, 93%, 94%, 95%, 96%, 97%, 98%, 99%, or 100% homology or sequence identity to SEQ ID NO: 32. In some embodiments, the polypeptide further comprises at least one P2A autocatalytic polypeptide cleavage site. In some embodiments, the polypeptide shares or comprises at least 90%, 91%, 92%, 93%, 94%, 95%, 96%, 97%, 98%, 99%, or 100% homology or sequence identity to SEQ ID NO: 33.

In some embodiments of any one of the polypeptides disclosed herein, the polypeptide further comprises an N-terminal IgE leader polypeptide sequence. In some embodiments, the N-terminal IgE leader polypeptide sequence shares or comprises at least 90%, 91%, 92%, 93%, 94%, 95%, 96%, 97%, 98%, 99%, or 100% homology or sequence identity to SEQ ID NO: 44. In some embodiments, the polypeptide shares or comprises at least 90%, 91%, 92%, 93%, 94%, 95%, 96%, 97%, 98%, 99%, or 100% homology or sequence identity to SEQ ID NO: 42.

In any of the polypeptides disclosed herein, the polypeptide may comprise any one or more of the SARS-CoV-2 polypeptides disclosed herein or otherwise conventionally known in the art. In some embodiments, the one or more SARS-CoV-2 polypeptides comprise an RBD polypeptide. In some embodiments, the RBD polypeptide is from the SARS-CoV-2 virus or a variant thereof. In some embodiments, the RBD polypeptide comprises a K417N, N439K, E484K, or N501Y mutation with reference to the full S protein (e.g., as set forth in NCBI Accession No. YP_009724390), or any combination thereof, or none of these mutations. In some embodiments, a nucleic acid encoding for the RBD polypeptide is represented by SEQ ID NO: 10 or 22. In some embodiments, the RBD polypeptide is represented by SEQ ID NO: 34. In some embodiments, the RBD polypeptide is an RBD tandem repeat single chain dimer polypeptide. In some embodiments, the RBD tandem repeat single chain dimer polypeptide comprises a K417N, N439K, E484K, or N501Y mutation with reference to the full S protein (e.g., as set forth in NCBI Accession No. YP_009724390), or any combination thereof, or none of these mutations. In some embodiments, a nucleic acid encoding the RBD polypeptide is represented by any one or more of SEQ ID NOs: 45, or 47-50. In some embodiments, the RBD polypeptide is represented by any one or more of SEQ ID NO: 46, or 52-55. In some embodiments, a nucleic acid encoding for an M polypeptide is represented by SEQ ID NOs: 11 or 23. In some embodiments, the M polypeptide is represented by SEQ ID NO: 35. In some embodiments, a nucleic acid encoding for an NP polypeptide is represented by SEQ ID NOs: 12 or 24. In some embodiments, the NP polypeptide is represented by SEQ ID NO: 36.

Any one of the polypeptides disclosed herein may be used in a medicament or for the manufacture of a medicament. In some embodiments, the medicament is used for the prevention, treatment, or inhibition of SARS-CoV-2 or other coronavirus in a subject. In some embodiments, the subject is a human.

Any one of the polypeptides disclosed herein may be recombinantly expressed. In some embodiments, the polypeptide is recombinantly expressed in a mammalian, bacterial, yeast, insect, or cell-free system.

### Methods of Therapy or Use

The terms “prime” and “boost” as used herein related to separate immunogenic compositions used in a heterologous prime-boost immunization approach. Immunizations or vaccines commonly require more than one administration of an immunogenic composition to induce a successful immunity against a target pathogen in a host. Compared to this homologous approach where the same composition is provided for all administrations, a heterologous prime-boost administration may be more effective in establishing robust immunity with greater antibody levels and improved clearing or resistance against some pathogens such as viruses, coronaviruses, SARS-CoV-2, bacteria, parasites, protozoa, helminths. In a heterologous prime-boost administration, at least one prime dose comprising one type of immunogenic composition is first provided. After the at least one prime dose is provided, at least one boost dose comprising another type of immunogenic composition is then provided. Administration of the at least one boost dose is performed at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the prime dose comprises a nucleic acid (e.g. DNA or RNA) that encodes for one or more antigens or epitopes, and the boost dose comprises a polypeptide that comprises one or more antigens or epitopes. In the host, the nucleic acid prime is translated in vivo to elicit an immune reaction and causes a greater response against the subsequent polypeptide boost.

In some embodiments, the nucleic acid prime comprises, consists essentially of, or consists of sequences from SARS-CoV-2 or other coronaviruses, including variants thereof. In some embodiments, the sequences from SARS-CoV-2 or other coronaviruses encode for an S, RBD, M, E, or NP polypeptide, including mutated or variant polypeptides thereof. In some embodiments, the nucleic acid prime also includes at least one HDAg sequence. In some embodiments, the nucleic acid sequences are codon optimized for expression in humans. In some embodiments, the polypeptide boost comprises, consists essentially of, or consists of polypeptides from SARS-CoV-2 or other coronaviruses. In some embodiments, the polypeptides from SARS-CoV-2 or other coronaviruses are S, RBD, M, E, or NP polypeptides. In some embodiments, the prime dose is a polypeptide, and the boost dose is a nucleic acid. General information about heterologous prime-boost approaches can be found in PCT Publications WO 2006/013106, WO 2006/040334, WO 2008/094188, each of which are hereby expressly incorporated by reference for the purpose of describing prime-boost methods.

Disclosed herein are immunogenic compositions or product combinations. In some embodiments, these immunogenic compositions or product combinations may be used in a prime-boost approach. In some embodiments, the immunogenic composition or product combination comprises (a) a nucleic acid comprising at least one nucleic acid sequence encoding a SARS-CoV-2 polypeptide, or (b) a polypeptide comprising at least one SARS-CoV-2 polypeptide, or both.

In some embodiments of any one of the immunogenic compositions or product combinations disclosed herein, the at least one nucleic acid sequence encoding for a SARS-CoV-2 polypeptide comprises i) a nucleic acid sequence encoding an RBD polypeptide; ii) a nucleic acid sequence encoding an NP polypeptide; iii) a nucleic acid sequence encoding an M polypeptide; iv) a nucleic acid sequence encoding an HDAg polypeptide; v) a nucleic acid sequence encoding a P2A autocatalytic polypeptide cleavage site; vi) a nucleic acid sequence encoding an IgE leader polypeptide; or vii) a nucleic acid sequence encoding a S polypeptide; or any combination thereof. In some embodiments, the nucleic acid is any one of the nucleic acids disclosed herein. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 1-12, which is optionally used in a medicament, such as for the prevention, treatment, or inhibition of SARS-CoV-2 in a subject, such as a mammal, preferably a human. In other embodiments, the nucleic acid is codon optimized for expression in a human. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 13-24, 39-40, 57-63, 71, 73, or 75, which is optionally used in a medicament, such as for the prevention, treatment, or inhibition of SARS-CoV-2 in a subject, such as a mammal, preferably a human. In some embodiments, the RBD polypeptide is an RBD tandem repeat single chain dimer. In some embodiments, the RBD polypeptide is from the SARS-CoV-2 virus or a variant thereof. In some embodiments, the RBD polypeptide comprises a K417N, N439K, E484K, or N501Y mutation with reference to the full S protein (e.g., as set forth in NCBI Accession No. YP_009724390), or any combination thereof, or none of these mutations. In some embodiments, the RBD polypeptide shares or comprises at least 90%, 91%, 92%, 93%, 94%, 95%, 96%, 97%, 98%, 99%, or 100% homology or sequence identity to any one or more of SEQ ID NO: 46, or 52-55. In some embodiments, the nucleic acid is provided in a recombinant vector. In some embodiments, the recombinant vector is pVAX1

In some embodiments of any one of the immunogenic compositions or product combinations disclosed herein, the at least one SARS-CoV-2 polypeptide comprises i) an RBD polypeptide sequence; ii) an NP polypeptide sequence; iii) an M polypeptide sequence; iv) an HDAg polypeptide sequence; v) a P2A autocatalytic polypeptide cleavage site sequence; vi) an IgE leader polypeptide sequence; or vii) an S polypeptide sequence; or any combination thereof. In some embodiments, the polypeptide is any one of the polypeptides disclosed herein. In some embodiments, the polypeptide shares or comprises at least 90%, 91%, 92%, 93%, 94%, 95%, 96%, 97%, 98%, 99%, or 100% homology or sequence identity to any one or more of SEQ ID NO: 25-36, 41-42, 64-70, 72, 74, or 76, which is optionally used in a medicament, such as for the prevention, treatment, or inhibition of SARS-CoV-2 in a subject, such as a mammal, preferably a human. In some embodiments, the RBD polypeptide is an RBD tandem repeat single chain dimer. In some embodiments, the RBD polypeptide is from the SARS-CoV-2 virus or a variant thereof. In some embodiments, the RBD polypeptide comprises a K417N, N439K, E484K, or N501Y mutation with reference to the full S protein (e.g., as set forth in NCBI Accession No. YP_009724390), or any combination thereof, or none of these mutations. In some embodiments, the RBD polypeptide shares or comprises at least 90%, 91%, 92%, 93%, 94%, 95%, 96%, 97%, 98%, 99%, or 100% homology or sequence identity to any one of SEQ ID NO: 46, or 52-55. In some embodiments, the polypeptide is recombinantly expressed. In some embodiments, the polypeptide is recombinantly expressed in a mammalian, bacterial, yeast, insect, or cell-free system.

In some embodiments, any one of the immunogenic compositions or product combinations disclosed herein further comprise an adjuvant. In some embodiments, the adjuvant is any adjuvant conventionally known in the art. In some embodiments, the adjuvant is alum and/or QS21.

Also disclosed herein are methods of generating an immune response and/or generating neutralizing antibodies in a subject using any one of the immunogenic compositions or product combinations disclosed herein. In some embodiments, these methods comprise administering to the subject at least one prime dose comprising the nucleic acid of any one of the immunogenic compositions or product combinations, and administering to the subject at least one boost dose comprising the polypeptide of any one of the immunogenic compositions or product combinations. In some embodiments, the immune response and/or neutralizing antibodies are against SARS-CoV-2 or other coronavirus. In some embodiments, the subject is a mammal, such as a mouse, rat, monkey, cat, dog, or human. In some embodiments, the at least one boost dose further comprises an adjuvant. In some embodiments, the adjuvant is any adjuvant conventionally known in the art. In some embodiments, the adjuvant is alum and/or QS21. In some embodiments, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the administration is provided enterally, orally, intranasally, parenterally, subcutaneously, intramuscularly, intradermally, or intravenously or any combination thereof, and optionally with in vivo electroporation. In some embodiments, the administration is performed in conjunction with an antiviral therapy. In some embodiments, the antiviral therapy comprises administration of dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof.

In some embodiments, administration of the nucleic acid prime and polypeptide boost comprising components of SARS-CoV-2 or other coronaviruses in a subject (e.g. mouse, rabbit, monkey, human) of any one of the immunogenic compositions or product combinations disclosed herein results in greater anti-S, anti-RBD, anti-M, anti-E, anti-NP, anti-SARS-CoV-2, or anti-coronavirus antibody titer at a ratio of 1, 2, 3, 4, 5, 6, 7, 8, 9, 10, 50, 100, 150, 200, 300, 400, 500, 600, 700, 800, 900, 1000, 5000, 10000, 100000, or 1000000 or any ratio within a range defined by any two of the aforementioned ratios compared to nucleic acid-only or polypeptide-only immunized, or unimmunized control organisms, quantified by techniques known in the art such as ELISA. In some embodiments, administration of the nucleic acid prime and polypeptide boost comprising components of SARS-CoV2 or other coronaviruses in a subject results in serum that neutralizes the in vitro or in vivo infectivity of SARS-CoV2 or other coronaviruses more effectively and reduces the incidence of infection or multiplicity of infection (MOI) to a ratio of 0.00001, 0.00005, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 or any ratio within a range defined by any two of the aforementioned ratios compared to sera from nucleic acid-only or polypeptide-only immunized, or unimmunized control organisms. In some embodiments, administration of the nucleic acid prime and polypeptide boost comprising components of SARS-CoV2 or other coronaviruses in a subject results in a greater number of interferon gamma (IFNγ)-positive cells (e.g. T cells, macrophages, natural killer (NK) cells) at a ratio of 1, 2, 3, 4, 5, 6, 7, 8, 9, 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, 1000, 5000, or 10000, or any ratio within a range defined by any two of the aforementioned ratios compared to nucleic acid-only or polypeptide-only immunized, or unimmunized control organisms.

Also disclosed herein are immunogenic compositions or product combinations for use in the treatment or inhibition of SARS-CoV-2 or other coronavirus. In some embodiments, the immunogenic compositions or product combinations comprise (a) a nucleic acid comprising at least one nucleic acid sequence encoding a SARS-CoV-2 polypeptide, or (b) a polypeptide comprising at least one SARS-CoV-2 polypeptide, or both. In some embodiments, the at least one nucleic acid sequence encoding a SARS-CoV-2 polypeptide comprises: i) a nucleic acid sequence encoding an RBD polypeptide; ii) a nucleic acid sequence encoding an NP polypeptide; iii) a nucleic acid sequence encoding an M polypeptide; iv) a nucleic acid sequence encoding an HDAg polypeptide; v) a nucleic acid sequence encoding a P2A autocatalytic polypeptide cleavage site; vi) a nucleic acid sequence encoding an IgE leader polypeptide; or vii) a nucleic acid sequence encoding a S polypeptide; or any combination thereof. In some embodiments, the nucleic acid is any one of the nucleic acids disclosed herein. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 1-12. In some embodiments, the nucleic acid is codon optimized for expression in a human. In some embodiments, the nucleic acid shares or comprises at least 90%, 91%, 92%, 93%, 94%, 95%, 96%, 97%, 98%, 99%, or 100% homology or sequence identity to any one or more of SEQ ID NO: 13-24, or 3940. In some embodiments, the at least one SARS-CoV-2 polypeptide comprises: i) an RBD polypeptide sequence; ii) an NP polypeptide sequence; iii) an M polypeptide sequence; iv) an HDAg polypeptide sequence; v) a P2A autocatalytic polypeptide cleavage site sequence; vi) an IgE leader polypeptide sequence; or vii) an S polypeptide sequence; or any combination thereof. In some embodiments, the polypeptide is any one of the polypeptides disclosed herein. In some embodiments, the polypeptide shares or comprises at least 90%, 91%, 92%, 93%, 94%, 95%, 96%, 97%, 98%, 99%, or 100% homology or sequence identity to any one or more of SEQ ID NO: 25-36, or 41-42. In some embodiments, the RBD polypeptide is an RBD tandem repeat single chain dimer. In some embodiments, the RBD polypeptide shares or comprises at least 90%, 91%, 92%, 93%, 94%, 95%, 96%, 97%, 98%, 99%, or 100% homology or sequence identity to SEQ ID NO: 46. In some embodiments, the polypeptide is recombinantly expressed. In some embodiments, the polypeptide is recombinantly expressed in a mammalian, bacterial, yeast, insect, or cell-free system. In some embodiments, the immunogenic composition or product combination further comprises an adjuvant. In some embodiments, the adjuvant is any adjuvant conventionally known in the art. In some embodiments, the adjuvant is alum and/or QS21. In some embodiments, the nucleic acid is provided in a recombinant vector. In some embodiments, the recombinant vector is pVAX1.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: Design of SARS-CoV-2 Immunogenic Composition Constructs

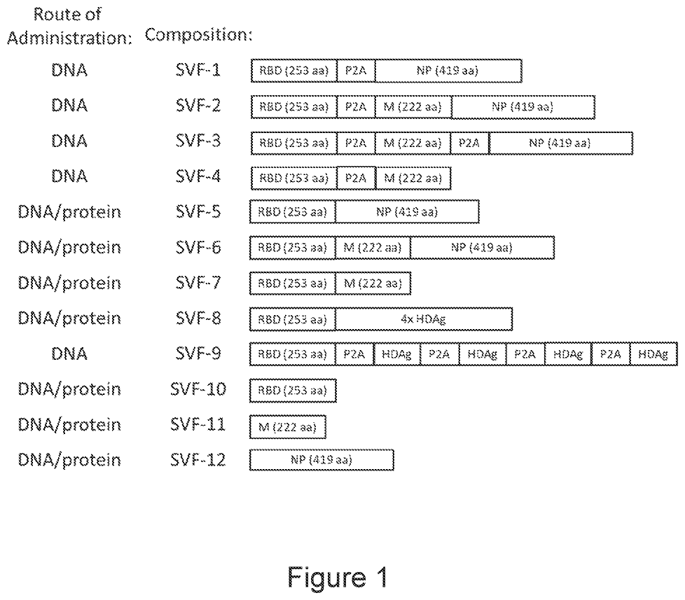

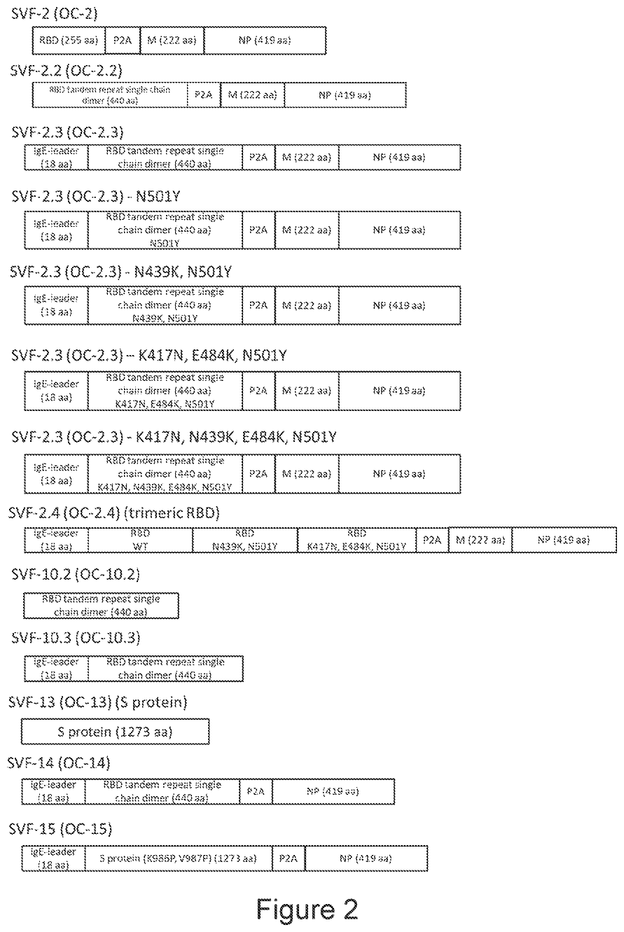

Several recombinant constructs containing components of the SARS-CoV-2 virus are prepared and are depicted in Table 1 and FIGS. 1-2. As the RBD of the S protein is known to be highly immunogenic, the majority of the constructs comprise an RBD sequence. In some cases, the RBD sequence is an RBD tandem repeat single chain dimer sequence. However, it is envisioned that a construct can have any combination of encoding sequences, in any order, from the SARS-CoV-2 virus or any other coronavirus. This includes constructs lacking an RBD sequence. This also includes sequences for coronavirus replication proteins or hemagglutinin esterase.

An RBD sequence can be found in SVF-1 (OC-1), SVF-2 (OC-2), SVF-3 (OC-3), SVF-4 (OC-4), SVF-5 (OC-4), SVF-6 (OC-6), SVF-7 (OC-7), SVF-8 (OC-8), SVF-9 (OC-9), SVF-10 (OC-10), SVF-14 (OC-14), SVF-2.2 (OC-2.2), SVF-2.3 (OC-2.3), and SVF-2.4 (OC-2.4) including any derivatives and/or mutants thereof.

An RBD tandem repeat single chain dimer is found in SVF-2.2 and SVF-2.3, and SVF-14 (OC-14), including any derivatives and/or mutants thereof.

A trimeric RBD construct is found in SVF-2.4, including any derivatives and/or mutants thereof.

A S protein sequence is found in SVF-13 (OC-13) and SVF-15 (OC-15), including any derivatives and/or mutants thereof.

An NP protein sequence is found in SVF-1, SVF-2, SVF-3, SVF-5, SVF-6, SVF-12 (OC-12), SVF-14, SVF-15, SVF-2.2, SVF-2.3, and SVF-2.4, including any derivatives and/or mutants thereof.

An M protein sequence is found in SVF-2, SVF-3, SVF-4, SVF-6, SVF-7, SVF-11 (OC-11), SVF-2.2, SVF-2.3, and SVF-2.4, including any derivatives and/or mutants thereof.

At least one P2A autocatalytic peptide cleavage site is found in SVF-1, SVF-2, SVF-3, SVF-4, SVF-9, SVF-14, SVF-15, SVF-2.2, SVF-2.3, and SVF-2.4, including any derivatives and/or mutants thereof. The presence of this P2A autocatalytic peptide cleavage site (which may trivially be substituted with another autocatalytic peptide cleavage site), allows for translation of separate proteins in the target cell from one or more contiguous nucleic acid gene or cassette. The presence of the autocatalytic peptide cleavage site also suggests that recombinant protein expression and purification of said constructs will lead to separate polypeptide components, which will be difficult to purify. While still possible (e.g. with the same or different epitope tags), using the other constructs for producing protein for immunogenic administration is more feasible.

In some embodiments, the recombinant constructs further contain components of the hepatitis B virus or hepatitis D virus. This is seen with SVF-8 and SVF-9, where HDAg copies of 4 different consensus sequences (genotypes 1 A, 1B, 2A, and 2B) are provided. HDAg is also a highly immunogenic polypeptide, and it is envisioned that inclusion of the HDAg sequences improves immunogenic response to the RBD or other coronavirus sequences. It is also envisioned that these constructs will provide dual immunogenic response against SARS-CoV-2 (or other coronavirus) and hepatitis B or D.

Constructs SVF-10 (RBD), SVR-11 (M), SVF-12 (NP), and SVF-13 (S) are provided as single SARS-CoV-2 sequence compositions to assess relative immunogenicity of the different components.

TABLE 1
SARS-COV-2 immunogenic composition candidates

| | Wild-type | Human codon | | |
|---|---|---|---|---|
| | DNA | optimized | Polypeptide | |
| Composition | sequence | sequence | sequence | Form |
| SVF-1 (OC-1) | SEQ ID NO: 1 | SEQ ID NO: 13 | SEQ ID NO: 25 | DNA |
| SVF-2 (OC-2) | SEQ ID NO: 2 | SEQ ID NO: 14 | SEQ ID NO: 26 | DNA |
| SVF-3 (OC-3) | SEQ ID NO: 3 | SEQ ID NO: 15 | SEQ ID NO: 27 | DNA |
| SVF-4 (OC-4) | SEQ ID NO: 4 | SEQ ID NO: 16 | SEQ ID NO: 28 | DNA |
| SVF-5 (OC-5) | SEQ ID NO: 5 | SEQ ID NO: 17 | SEQ ID NO: 29 | DNA or protein |
| SVF-6 (OC-6) | SEQ ID NO: 6 | SEQ ID NO: 18 | SEQ ID NO: 30 | DNA or protein |
| SVF-7 (OC-7) | SEQ ID NO: 7 | SEQ ID NO: 19 | SEQ ID NO: 31 | DNA or protein |
| SVF-8 (OC-8) | SEQ ID NO: 8 | SEQ ID NO: 20 | SEQ ID NO: 32 | DNA or protein |
| SVF-9 (OC-9) | SEQ ID NO: 9 | SEQ ID NO: 21 | SEQ ID NO: 33 | DNA |
| SVF-10 (OC-10) | SEQ ID NO: 10 | SEQ ID NO: 22 | SEQ ID NO: 34 | DNA or protein |
| SVF-11 (OC-11) | SEQ ID NO: 11 | SEQ ID NO: 23 | SEQ ID NO: 35 | DNA or protein |
| SVF-12 (OC-12) | SEQ ID NO: 12 | SEQ ID NO: 24 | SEQ ID NO: 36 | DNA or protein |
| SVF-2.2 (OC-2.2) | N/A | SEQ ID NO: 39 | SEQ ID NO: 41 | DNA |
| SVF-2.3 (OC-2.3) | N/A | SEQ ID NO: 40 | SEQ ID NO: 42 | DNA |
| SVF-2.3 (OC-2.3)- | N/A | SEQ ID NO: 57 | SEQ ID NO: 64 | DNA |
| N501Y | | | | |
| SVF-2.3 (OC-2.3)- | N/A | SEQ ID NO: 58 | SEQ ID NO: 65 | DNA |
| N439K, N501Y | | | | |
| SVF-2.3 (OC-2.3 - | N/A | SEQ ID NO: 59 | SEQ ID NO: 66 | DNA |
| K417N, E484K, | | | | |
| N501Y | | | | |
| SVF-2.3 (OC-2.3)- | N/A | SEQ ID NO: 60 | SEQ ID NO: 67 | DNA |
| K417N, N439K, | | | | |
| E484K, N501Y | | | | |
| SVF-2.4 (OC-2.4) | N/A | SEQ ID NO: 61 | SEQ ID NO: 68 | DNA |
| SVF-14 | N/A | SEQ ID NO: 62 | SEQ ID NO: 69 | DNA |
| SVF-15 | N/A | SEQ ID NO: 63 | SEQ ID NO: 70 | DNA |
| SVF-13 (OC-13) | N/A | SEQ ID NO: 71 | SEQ ID NO: 72 | DNA or protein |
| SVF-10.2 (OC-10.2) | N/A | SEQ ID NO: 73 | SEQ ID NO: 74 | DNA or protein |
| SVF-10.3 (OC-10.3) | N/A | SEQ ID NO: 75 | SEQ ID NO: 76 | DNA or protein |

### Example 2: Methodology

### Animals

BALB/c, C57BL/6 and K18-hACE2 (B6.Cg-Tg(KI8-ACE2)2Prlmn/J) mice can be obtained from the Jackson Laboratory. All mice are 8-10 weeks old at the start of the experiments and maintained under standard conditions. New Zealand White rabbits are purchased from commercial vendors.

### Recombinant Vectors

Sequences for SARS-CoV-2 are obtained from NCBI GenBank accession number: MN908947.3 (e.g. complete genome), YP_009724390 (e.g. surface glycoprotein), YP_009724393.1 (e.g. membrane glycoprotein), and YP_009724397.2 (e.g. nucleocapsid phosphoprotein). The HDAg sequences of genotypes 1 and 2 are obtained from four different clinical isolates; US-2 and CB, and 7/18/83 and TW2476, respectively, and codon optimized for expression in human.

For the DNA immunogenic compositions, genes are cloned into the pVAX1 backbone (ThermoFisher) using restriction sites BamHI and XbaI. Plasmids are grown in TOP10 E. coli cells (ThermoFisher) and purified for in vivo injections using Qiagen Endofree DNA purification kit (Qiagen GmbH) following manufacturer's instructions. The correct gene size is confirmed by restriction enzyme digests. In addition, all cloned gene sequences were sequenced to confirm the correct nucleotide sequence.

For protein expression constructs, genes are cloned into the pET100 E. coli T7 expression vector (ThermoFisher). Other commercially available expression vectors can be used. Expression vectors are transformed into BL21(DE3) E. coli (or other T7 expression E. coli strain) and induced for purification according to protocols known in the art.

### Western Blot

Western blot is performed as known in the art. HeLa cells are transfected with each pVAX1 construct using Lipofectamine 3000 Transfection Reagent (ThermoFisher). A pVAX1 plasmid with a GFP reporter gene is used as a control. For protein detection, serum from rabbits immunized with one of the SARS-CoV-2 pVAX1 compositions or commercially available anti-SARS-CoV-2 antibodies, and an appropriate HRP secondary antibody, are used. Chemiluminescence is induced with the Pierce™ ECL Plus Western Blotting Substrate and images are collected with a Gel Doc XR+ System (BioRad).

### Immunization Protocols

To evaluate the immunogenicity of the constructs in vivo, mice and rabbits are immunized at monthly intervals and sacrificed two weeks later for spleen and blood collection. In brief, mice (five to ten per group) are immunized intramuscularly (i.m.) in the tibialis cranialis anterior (TA) muscle with 1-50 μg plasmid DNA in a volume of 30-50 μL in sterile PBS by regular needle (27G) injection followed by in vivo electroporation (EP) using the Cliniporator2 device (IGEA, Carpi, Italy). During in vivo electroporation, a 1 ms 600 V/cm pulse followed by a 400 ms 60 V/cm pulse pattern is used to facilitate better uptake of the DNA. Prior to vaccine injections, mice are given analgesic and kept under isoflurane anesthesia during the vaccinations. For studies in rabbits, 2-4 New Zealand White rabbits per group are immunized with 100 μg to 900 μg plasmid DNA. Vaccines are administered by i.m. injection in 300 μL sterile PBS to the right TA muscle followed by in vivo EP.

### Detection of IFNγ Cells by Enzyme-Linked Immunospot Assay (ELISpot)

Two weeks after the last immunization, splenocytes from each immunized group of mice pooled are collected and tested for their ability to induce SARS-CoV-2-specific T cells based on IFN-γ secretion for 48 h as known in the art using SARS-CoV-2 derived peptides and/or proteins in a commercially available ELISpot assay (Mabtech, Nacka Strand, Sweden).

### Antibody Detection by ELISA

Detection of mouse and rabbit IgG against various SARS-CoV-2 peptides and/or proteins is performed using protocols known in the art. Antibody titers are determined as endpoint serum dilutions at which the OD value (e.g. at 405 nm or 492 nm) is at least twice the OD of the negative control (non-immunized or control animal serum) at the same dilution.

### In Vitro SARS-CoV-2 Neutralization Assay

Neutralization ability of immunization sera from animals is assessed in vitro. Vero E6 cells are grown to confluence on a culture plate. Media containing either sera from animals immunized with the SARS-CoV-2 compositions, or sera from control animals, is added to the cells. The cells are then infected with SARS-CoV-2 virus particles. Viral infectivity and serum neutralization are assessed by counting viral plaques or viral titer by detection of the viral genome/gene(s).

In Vivo SARS-CoV-2 Neutralization Assay in hACE2 Mouse Model

Wild-type or K18-hACE2 mice are immunized with the SARS-CoV-2 immunogenic compositions or a control. Different combinations are employed, including but not limited to DNA-only compositions, protein-only compositions, DNA prime/protein boost compositions, or protein-prime/DNA-boost compositions. K18-hACE2 mice are then infected with SARS-CoV-2 virus particles. For wild-type mice, they were made transiently transgenic for hACE2 by hydrodynamic injection, or other relevant techniques, 1-5 days prior to infection with SARS-CoV-2. Effect of the viral infection, including mouse weight, symptoms, morbidity and mortality, and viral load, are assessed.

### Statistical Analysis

Data are analyzed using GraphPad Prism V.5 and V.8 software and Microsoft Excel V.16.13.1.

### Example 3: SARS-CoV2 DNA and Protein Compositions are Immunogenic in Animals

While immunogenic compositions and vaccines have traditionally been either whole organisms or antigenic proteins, it has been recently shown that in vivo administration of DNA to living tissue and the subsequent transcription and translation of antigenic proteins are also highly effective in triggering an immune response. These DNA prime/protein boost immunogenic compositions are being explored as potential vaccine candidates against various diseases.

Mice are immunized with (1) a DNA composition comprising one of the compositions disclosed herein (3 sequential doses of 50 μg DNA), (2) a polypeptide composition comprising one of the compositions disclosed herein (3 sequential does of 20 μg protein with alum adjuvant), or (3) a DNA composition comprising one of the compositions disclosed herein followed by a polypeptide composition comprising one of the compositions disclosed herein (2 doses of 50 μg DNA then 2 doses of 20 μg protein with alum).

Following 1, 2, 3, 4, 5, 6, or 7 days, or 1, 2, 3, 4, 5, 6, 7, 8, 9, or 10 weeks, or 1, 2, 3, 4, 5, 6, 7, 8, 9, 10, 11, 12 months or any time within a range defined by any two of the aforementioned times as the duration of administration of the DNA prime/protein boost compositions, immunity of the mice against SARS-CoV-2 antigens is assessed. White blood cells are purified from mouse whole blood samples and incubated with purified polypeptide antigens, including S protein, RBD, M protein, and NP protein. Cells are also incubated with Concanavalin A (“ConA”) as a positive control, and two ovalbumin peptides (“OVA Th” and “OVA CTL”) as negative controls. The population frequency of interferon gamma (IFNγ) producing cells in response to antigen exposure is assessed by enzyme-linked immunospot assay (ELISpot). Briefly, white blood cells are incubated with antigen in wells coated with IFNγ antibodies. The cells are then removed, and biotinylated IFNγ antibodies, alkaline phosphatase-crosslinked streptavidin, and alkaline phosphatase substrate colorimetric reagents are added to the wells in succession with thorough washing in between. The plate is then allowed to dry and the remaining colored spots that correspond to IFNγ-secreting cells are counted by microscopy.

Treated mice show a comparatively stronger immune cell response overall. This demonstrates that this DNA prime/protein boost approach may be effective at inducing a robust immunogenic response greater than traditional protein or organism-based compositions for certain pathogens.

Corresponding experiments are also performed in rabbits (Oryctolagus cuniculus). New Zealand white rabbits are immunized with (1) a DNA-only composition comprising one of the compositions disclosed herein, (2) a protein-only composition comprising one of the compositions disclosed herein, or (3) a DNA prime/protein boost composition comprising one or more of the compositions disclosed herein. Compositions are administered four times as weeks 0, 4, 8, and 12, with either 900 μg DNA im/EP or 300 μg protein with alum administered for each dose. For DNA-protein compositions (3), 900 μg DNA im/EP is administered for the first dose at week 0, and 300 μg protein with alum is administered for the second, third, and fourth doses at weeks 4, 8, and 12. Anti-RBD titers in sera are assessed at weeks 0, 2, 10, and 14 (i.e. 2 weeks after each dosage). Not only does the DNA prime/protein boost composition (3) result in greater overall titers compared to DNA-only (1) and protein-only (2) compositions, but also induces robust antibody production more rapidly, by week 2, relative to the protein-only composition.

Active immunization using the immunogenic compositions described herein is able to induce functional T cells to SARS-CoV-2 or coronavirus antigens.

### Example 4: Immunogenic DNA Compositions Induce Production of SARS-CoV-2 Neutralizing Antibodies in Animals

A single 50 μg dose of DNA expression cassettes comprising compositions SVF-2, SVF-2.2, SVF-2.3, or only spike protein (as a control) was administered to BALC/c and C57BL/6 mice. Serum samples from the test mice were obtained two weeks following administration, and the presence of neutralizing antibodies specific for SARS-CoV-2 protein components was assessed by ELISA (end point titer) and in vitro neutralization assay. Results are shown below in Tables 2 (BALB/c) and 3 (C57BL/6). Composition SVF-2.3 resulted in the production of anti-SARS-CoV-2 spike protein antibodies comparable with the composition of only spike protein, but also conferred immunogenicity against SARS-CoV-2 nucleoprotein in BALB/c mice. Serum from BALB/c mice treated with composition SVF-2.3 also successfully neutralized SARS-CoV-2 infection in an in vitro assay. (S=spike protein; RBD=receptor binding domain; NP=nucleoprotein). The same responses are shown two weeks after a second immunization administered three weeks after the first immunization (Tables 4 and 5).

TABLE 2
Quantification of BALB/c mice
serum after DNA composition administration

| | Anti-S | Anti-RBD | Anti-NP | Neutralization |
|---|---|---|---|---|
| Composition | ELISA | ELISA | ELISA | SARS-COV-2 |
| SVF-2 | <60 | <60 | 2160 | <8 |
| SVF-2.2 | 60 | 60 | 1080 | <8 |
| SVF-2.3 | 1440 | 720 | 1080 | 8 |
| S DNA | 1440 | 360 | <60 | 8 |

TABLE 3
Quantification of C57BL/6 mice serum after
DNA composition administration

| | Anti-S | Anti-RBD | Anti-NP | Neutralization |
|---|---|---|---|---|
| Composition | ELISA | ELISA | ELISA | SARS-COV-2 |
| SVF-2 | <60 | <60 | 360 | <8 |
| SVF-2.2 | <60 | <60 | 135 | <8 |
| SVF-2.3 | 360 | 210 | <60 | <8 |
| S DNA | 360 | 360 | <60 | 8 |

TABLE 4
Quantification of BALB/c mice serum after
2 rounds of DNA composition administration

| | Anti-S | Anti-RBD | Anti-NP | Neutralization |
|---|---|---|---|---|
| Composition | ELISA | ELISA | ELISA | SARS-COV-2 |
| SVF-2 | <60 | <60 | not tested | <8 |
| SVF-2.2 | 60 | 720 | not tested | <8 |
| SVF-2.3 | 51480 | 64800 | not tested | 256 |
| S DNA | 12960 | 12960 | not tested | 256 |

TABLE 5
Quantification of C57BL/6 mice serum
after 2 rounds of DNA composition administration

| | Anti-S | Anti-RBD | Anti-NP | Neutralization |
|---|---|---|---|---|
| Composition | ELISA | ELISA | ELISA | SARS-COV-2 |
| SVF-2 | <60 | <60 | not tested | <8 |
| SVF-2.2 | <60 | <60 | not tested | <8 |
| SVF-2.3 | 36360 | 8280 | not tested | 128 |
| S DNA | 25960 | 25960 | not tested | 512 |

### Example 5: Additional Exemplary Constructs are Immunogenic in Mice

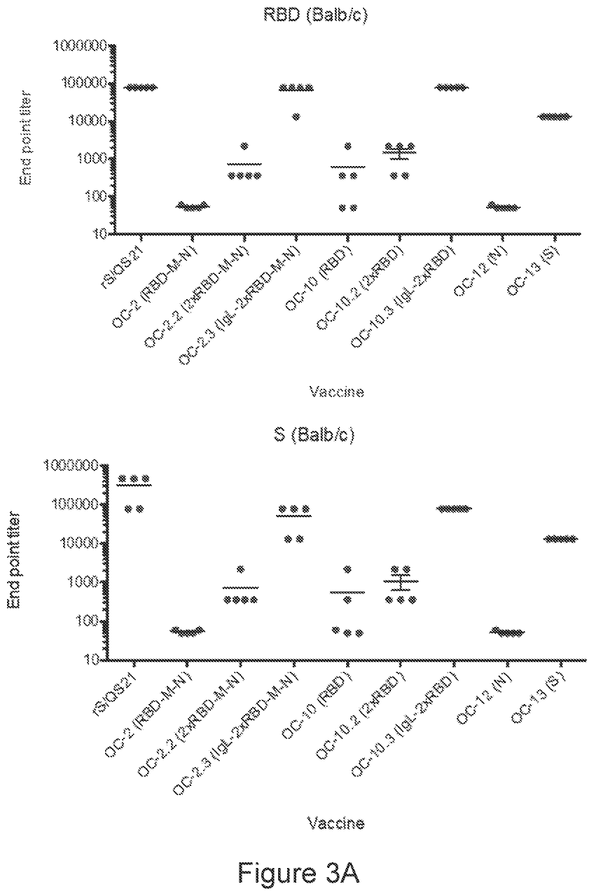

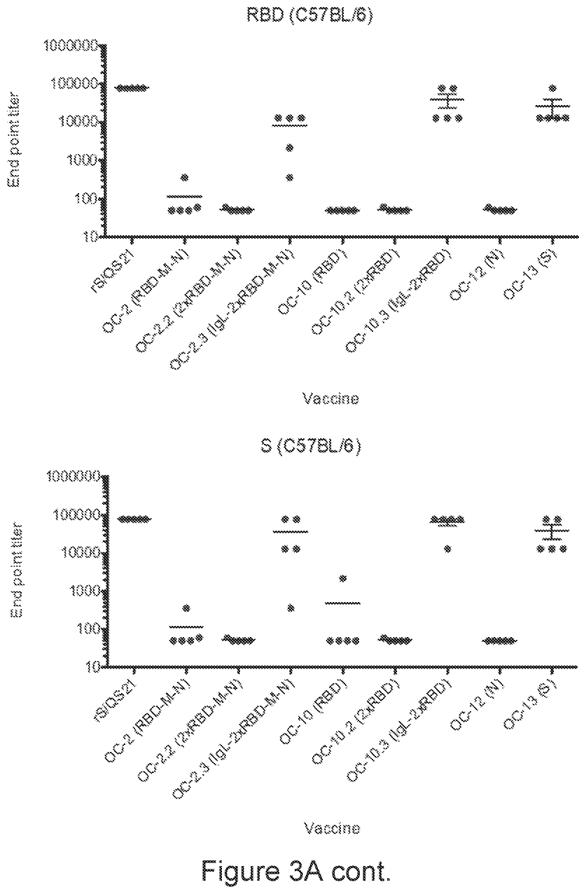

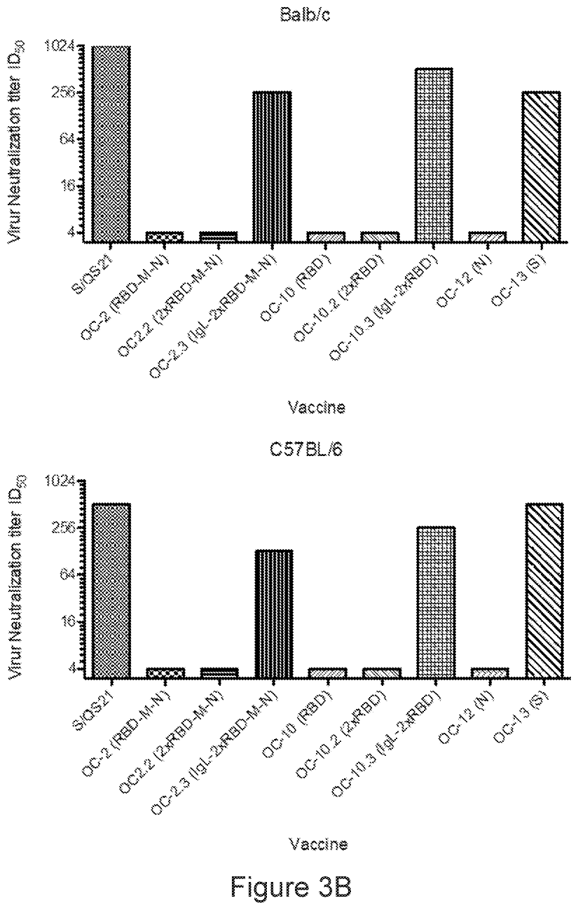

BALB/c and C57BL16 mice were immunized at weeks 0 and 3 with 50 μg of plasmid construct DNA using in vivo EP. The constructs used were OC-2, OC-2.2, OC-2.3, OC-10, OC-10.2, OC-10.3, OC-12, and OC-13, with recombinant S protein with QS21 adjuvant used as control. Serum samples from the test mice were obtained two weeks following administration of the second dose, and the presence of neutralizing antibodies specific for SARS-CoV-2 RBD and S protein was assessed by ELISA (FIG. 3A). Levels are given as the end point titer defined as the highest dilution giving an optical density at 450 nm of twice the negative control at the same dilution. Constructs OC-2.3, OC-10.3, and OC-13 exhibited robust immunogenic properties in both BALB/c and C57BL/6 mice.

The in vitro neutralization of immunized mice serum against SARS-CoV-2 was assessed. Pooled serum samples from each group of mice were incubated with SARS-CoV-2 and then added to Vero-E6 cells. The level of viral cytopathic effect (CPE) was determined by inspection under microscope and the virus neutralization titer ID₅₀ was determined as the dilution of serum giving 50% inhibition of CPE (FIG. 3B). Mice immunized with constructs OC-2.3, OC-10.3, and OC-13 resulted in serum that robustly neutralized SARS-CoV-2 infectivity.

### Example 6: The Immunogenic Compositions Induce T Cell Responses in Mice

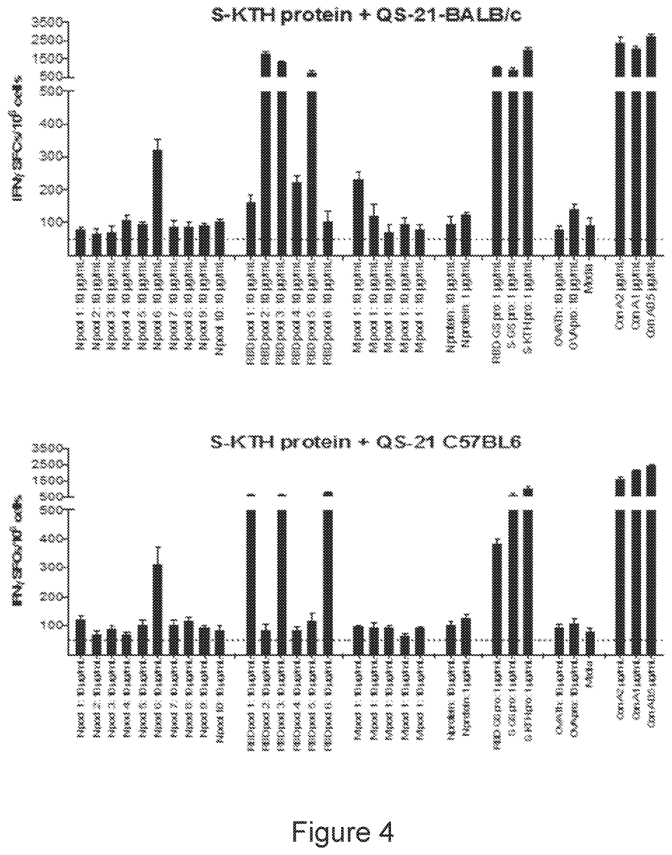

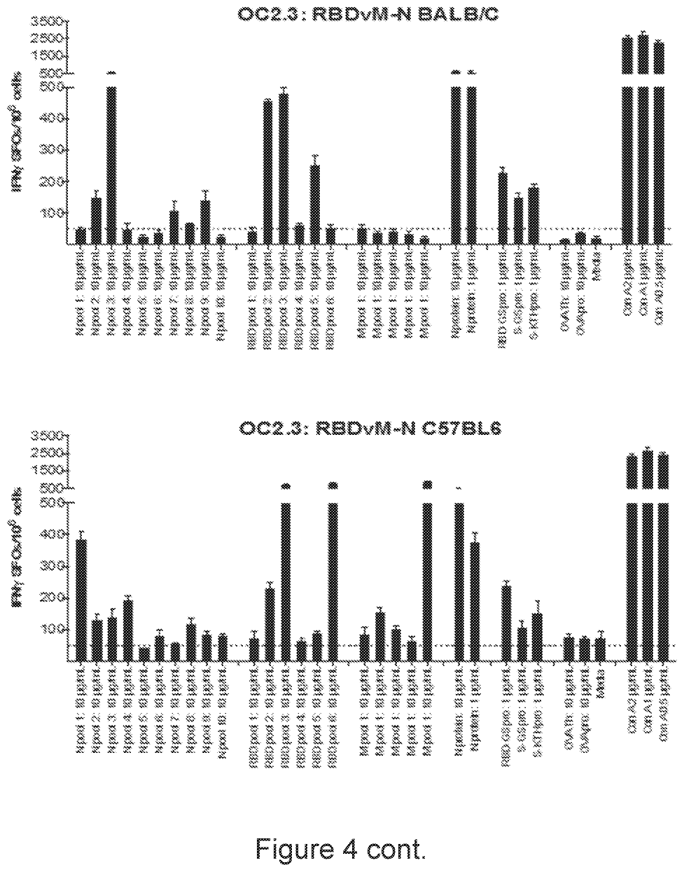

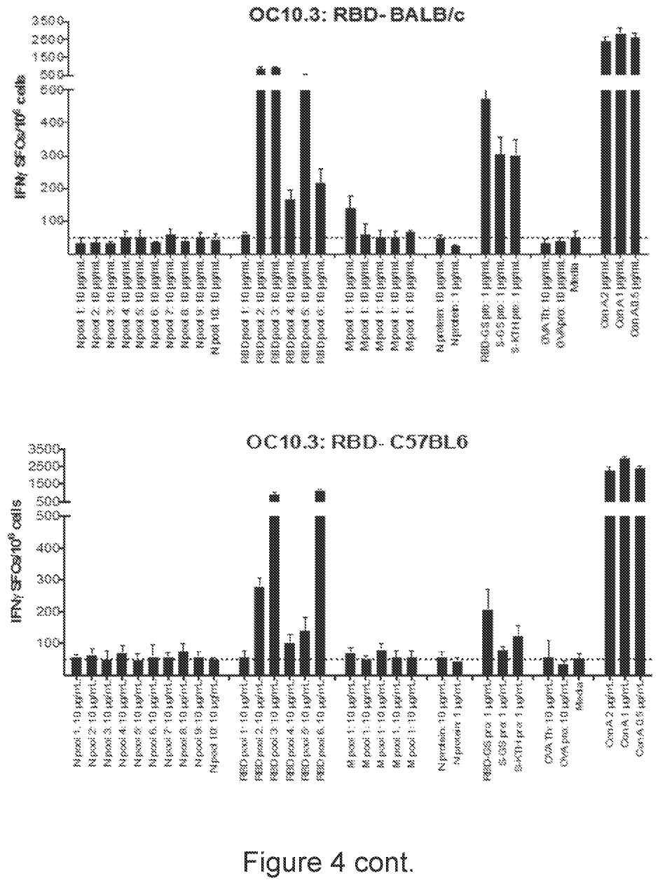

BALB/c and C57BL/6 mice were immunized at weeks 0 and 3 with 50 μg of OC-2.3 and OC-10.3 construct DNA using in vivo EP, with recombinant S protein with QS21 adjuvant as control. Responses of the mice T cells against peptide pools spanning the RBD, M, and NP proteins was detected by interferon gamma ELISpot (FIG. 4). “S-KTH” indicates recombinant S protein provided by Royal Technical University (KTH). “S-GS” indicates recombinant S protein obtained from Genscript (#Z03501). “RBD-GS” indicates recombinant RBD of S protein obtained from Genscript (#Z03479). These peptide pools were generated as 20 amino acid long peptides with 10 amino acids overlap. Ovalbumin peptides were used as negative control, and concanavalin A was used as positive control. Mice immunized with OC-2.3, which contains sequences for RBD, M, and NP protein, resulted in robust T cell activation against RBD and N peptides and protein, while M peptides were less reactive. Mice immunized with OC-10.3, which comprises only RBD, resulted in robust T cell activation only against RBD peptides and protein.

### Example 7: Sera from Immunized Animals are Effective at Neutralizing SARS-CoV-2 Infection

The ability of induced antibodies to neutralize SARS-CoV-2 infection in vivo is further determined using the K18-hACE2 mice model or transiently hACE2-transgenic wild-type mice. Total IgG is purified from immunized and non-immunized rabbits and is injected in mice. The DNA prime/protein boost-induced antibodies protects, or significantly delays peak viremia in all challenged mice better than the DNA-only or protein-only compositions.

### Example 8: T Cell Response Against SARS-CoV-2 Epitopes can be Enhanced with a Prime/Boost Approach

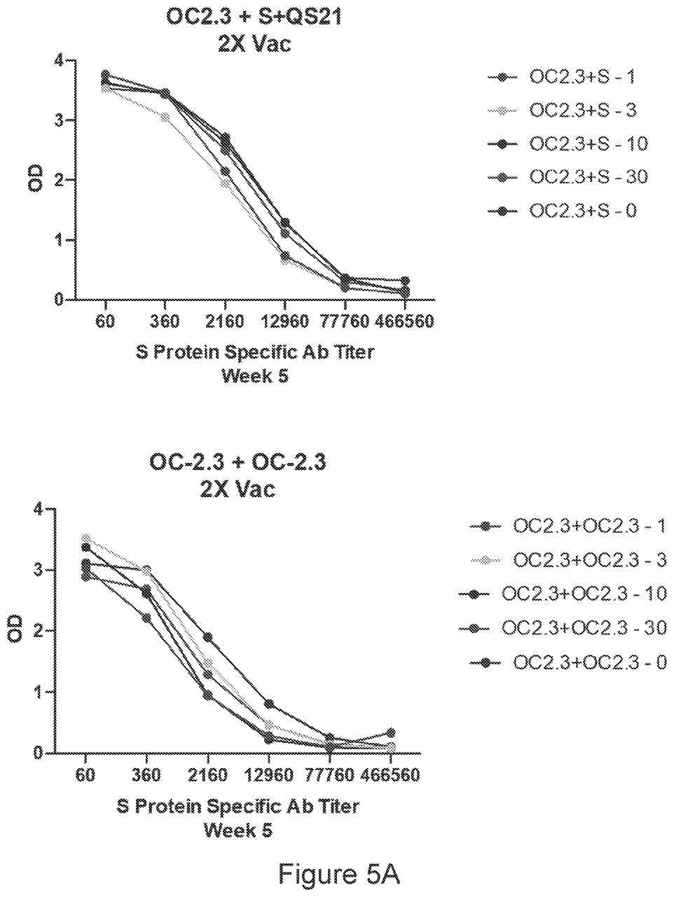

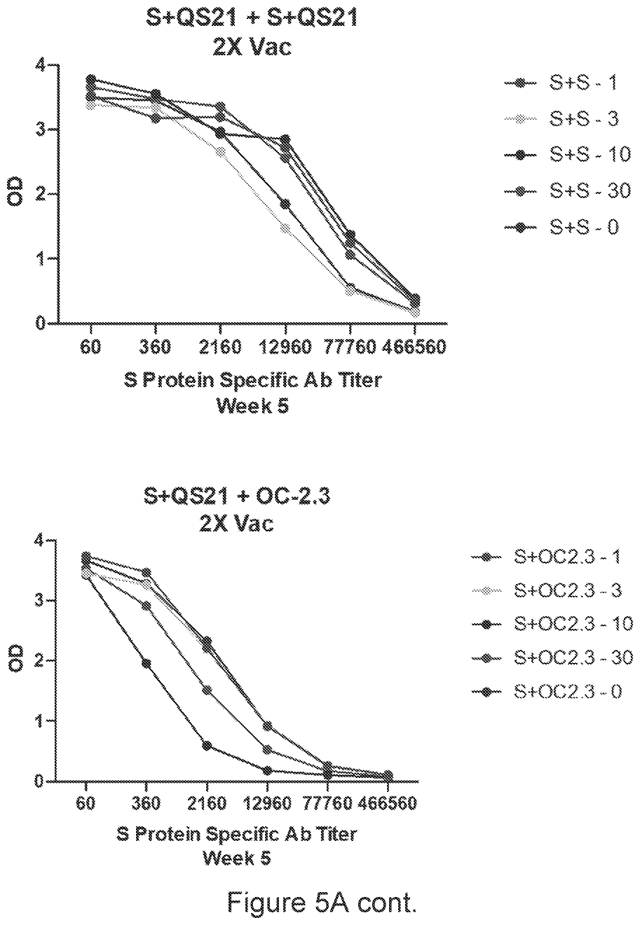

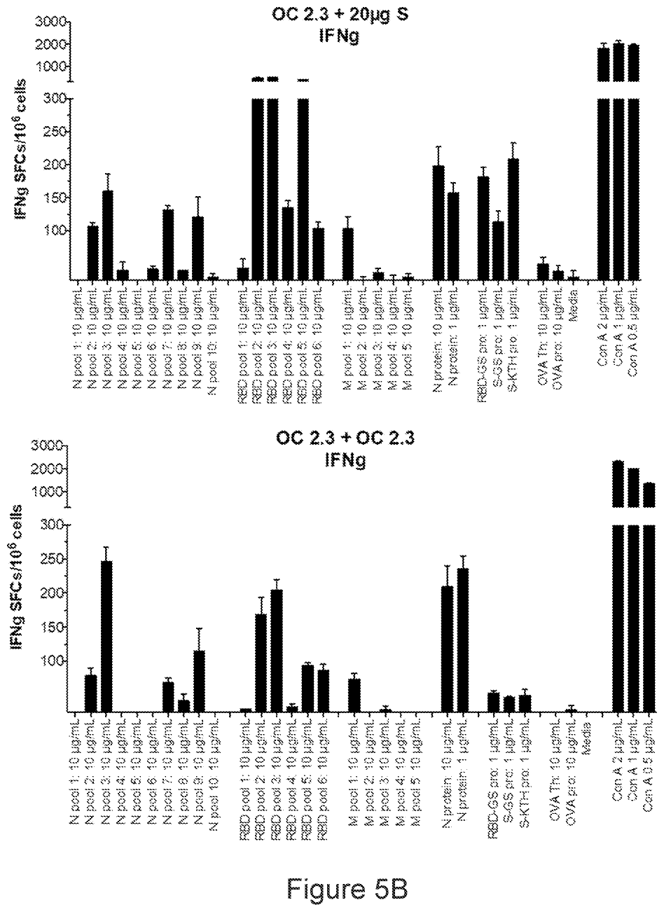

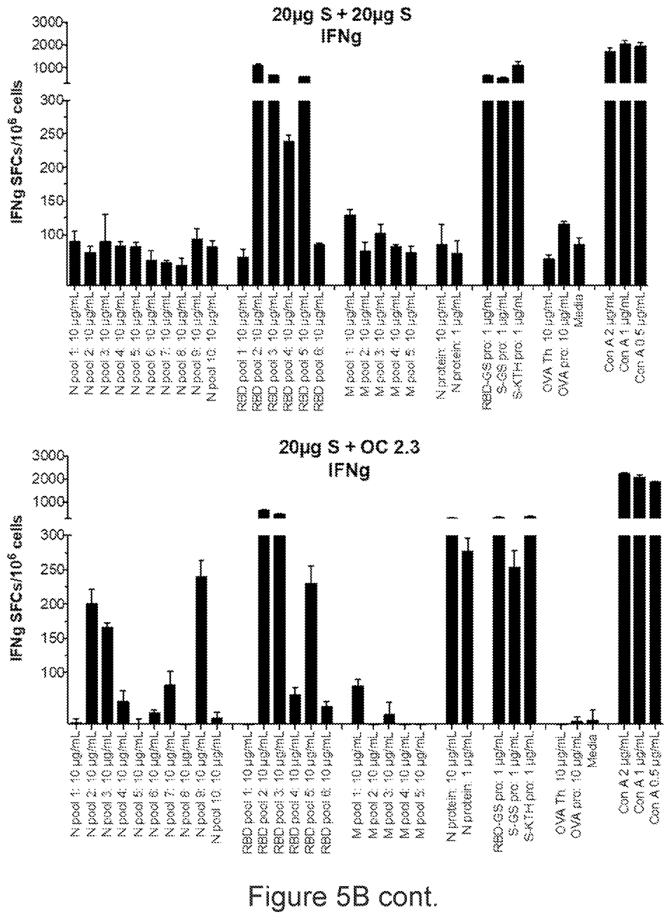

The effects of homologous (DNA only prime and boost; or protein only prime and boost) and heterologous priming (DNA prime, protein boost; or protein prime, DNA boost) with the OC-2.3 DNA construct and recombinant S protein with QS21 adjuvant (rS/QS21) was tested in BALB/c mice. The mice were immunized at weeks 0 and 3 with either 50 μg of plasmid construct DNA using in vivo EP, or recombinant S protein with QS21 adjuvant. FIG. 5A shows the anti-S protein titers in serum from in immunized mice (5 mice tested, labeled “0”, “1”, “3”, “10”, and “30”). Each of the 4 conditions (i.e. different combinations of S/QS21 peptide and OC-2.3 DNA as either prime or boost, or both). FIG. 5B shows T cell responses from the immunized mice towards peptide pools spanning the SARS-CoV-2 RBD, M, and NP proteins as detected by ELISpot. These peptide pools were generated as 20 amino acid long peptides with 10 amino acids overlap. Ovalbumin peptides were used as negative control, and concanavalin A was used as positive control. As seen for both the OC-2.3 DNA prime and rS/QS21 boost approach and the rS/QS21 prime and OC-2.3 DNA prime approach, the heterologous combination results in robust immunogenicity against RBD protein and peptides while also resulting in reactivity towards NP peptides and protein. This improved coverage of the SARS-CoV-2 viral components will provide improved protection against the virus as well as various strains or mutants where a certain component is conserved.

### Example 9: The Immunogenic Compositions are Immunogenic in Rabbits and Non-Human Primates

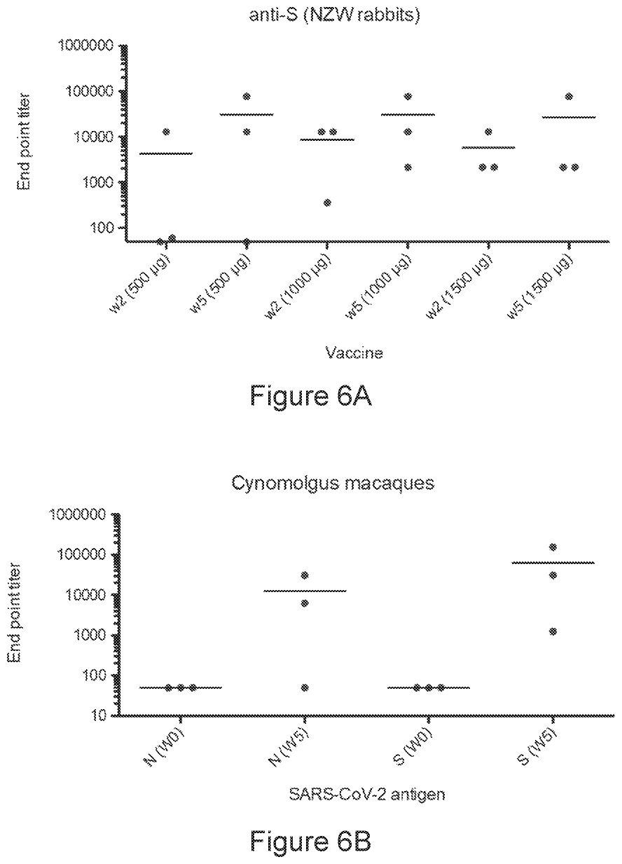

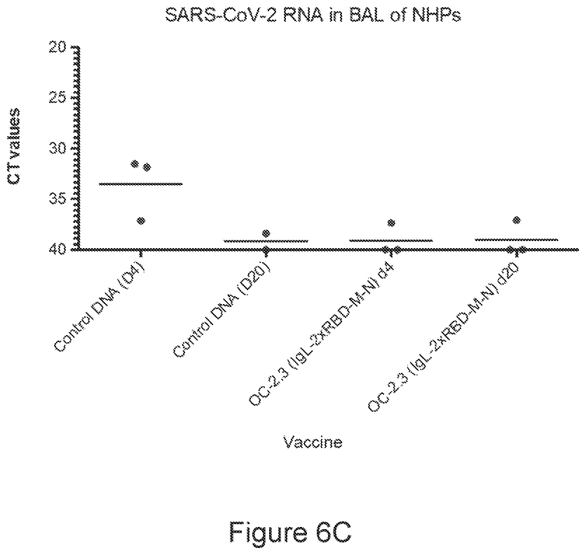

The immunogenic abilities of the OC-2.3 DNA construct in rabbits and cynomolgus macaques was assessed. The rabbits were administered with either 500, 1000, or 1500 μg of OC-2.3 DNA using in vivo EP at weeks 0 and 3. The macaques were administered with 1000 μg of OC-2.3 DNA using in vivo EP at weeks 0 and 3. The injection was performed using a single step procedure using a custom injection device. The anti-S antibody levels in the animals were assessed after the second administration (FIGS. 6A-B). Levels are given as the end point titer defined as the highest dilution giving an optical density at 450 nm of twice the negative control at the same dilution.

Cynomolgus macaques (groups of 3) were immunized with 1000 μg OC-2.3 or control DNA (HBV DNA) as two doses at weeks 0 and 3, and subsequently challenged with SARS-CoV-2 (0.5 mL intranasally and 4.5 mL intratracheally with 10⁶ pfu/mL). Bronchoalveolar lavage (BAL) samples were taken at days 4 and 20 post-challenge, and SARS-CoV-2 RNA was quantified by qPCR (FIG. 6C). A Ct value greater than 40 represents RNA levels below the detection limit. Monkeys immunized with OC-2.3 showed essentially undetectable levels of SARS-CoV-2 RNA at both days 4 and 20, whereas monkeys immunized with control DNA exhibited a detectable SARS-CoV-2 infection at day 4, and clearance of the infection by day 20. Quantification of antibody titers and presence of SARS-CoV-2 RNA in BAL is provided in Table 6. Leakage was noted with immunizations on subjects 4 and 5.

TABLE 6
Quantification of tested Cynomolgus macaques

| | | | | SARS-COV-2 DNA | | |
|---|---|---|---|---|---|---|
| | Control DNA (HBV) | | | (IgL-2xRBD-M-N; OC-2.3) | | |
| Histological finding | Subject 1 | Subject 2 | Subject 3 | Subject 4 | Subject 5 | Subject 6 |
| Anti-S titer | <50 | <50 | <50 | 31250 | 1250 | 156250 |
| Anti-HBV PreS1 titer | 50 | 31250 | 31250 | <50 | <50 | <50 |
| SARS-COV-2 RNA in | 31.52 | 31.86 | 37.14 | 37.34 | >40 | >40 |
| BAL day 4 (Ct value) | | | | | | |
| SARS-COV-2 RNA in | 38.39 | >40 | Not | >40 | 37.04 | >40 |
| BAL day 20 (Ct value) | | | tested | | | |

### Example 10: Human Clinical Trials with an Exemplary Immunogenic Composition Candidate

The following example describes embodiments of using an immunogenic composition or product combination, optionally comprised of a nucleic acid component and a polypeptide component, used to treat or prevent viral infections caused by coronaviruses such as SARS-CoV-2.

The DNA prime/protein boost compositions are administered to human patients enterally, orally, intranasally, parenterally, subcutaneously, intramuscularly, intradermally, or intravenously. These human patients may be currently infected with SARS-CoV-2, previously infected with SARS-CoV-2, at risk of being infected with SARS-CoV-2, or uninfected with SARS-CoV-2.

The DNA prime doses are administered first, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. After the first DNA prime dose, 1, 2, 3, 4, or 5 additional DNA prime doses can be administered 1, 2, 3, 4, 5, 6, 7, 8, 9, 10, 11, 12, 24, 36, or 48 days or weeks or any time within a range defined by any two of the aforementioned times after administration of the previous DNA prime dose, e.g., within 1-48 days or 1-48 weeks. The protein boost doses are administered following the DNA prime doses, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. The first protein boost dose is administered 1, 2, 3, 4, 5, 6, 7, 8, 9, 10, 11, 12, 24, 36, or 48 days or weeks or any time within a range defined by any two of the aforementioned times after administration of the final DNA prime dose. After the first protein boost dose, 1, 2, 3, 4, or 5 additional protein boost doses can be administered 1, 2, 3, 4, 5, 6, 7, 8, 9, 10, 11, 12, 24, 36, or 48 days or weeks or any time within a range defined by any two of the aforementioned times after administration of the previous protein boost dose.

Patients will be monitored for successful response against SARS-CoV-2, for example, production of anti-S protein, anti-RBD, anti-M protein, anti-NP protein, anti-SARS-CoV2 or anti-coronavirus antibodies. In the conditions where HDAg sequences are included, anti-HDAg antibodies in sera is also tested. Also expected is the rapid activation of T cells and other immune cells when exposed to SARS-CoV-2 or coronavirus antigens, and protection against future infections by SARS-CoV-2 or coronavirus.

In patients currently infected, previously infected, or at risk for infection SARS-CoV-2 or coronavirus, administration of the DNA prime/protein boost compositions may be performed in conjunction with antiviral therapy. Potential antiviral therapy therapeutics include but are not limited to dexamethasone, favipiravir, favilavir, remdesivir, tocilizumab, galidesivir, sarilumab, lopinavir, ritonavir, darunavir, ribavirin, interferon-α, pegylated interferon-α, interferon alfa-2b, convalescent serum, or any combination thereof. Patients will be monitored for side effects such as dizziness, nausea, diarrhea, depression, insomnia, headaches, itching, rashes, fevers, or other known side effects of the provided antiviral therapeutics.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A nucleic acid comprising at least one nucleic acid sequence encoding a SARS-CoV-2 polypeptide and at least one nucleic acid sequence encoding a P2A autocatalytic polypeptide cleavage site.

2-105. (canceled)
